(19) United States
(12) Patent Application Publication
(10) Pub. No.: US 2021/0117869 A1
(43) Pub. Date: Apr. 22, 2021

(54) ENSEMBLE MODEL CREATION AND SELECTION

(71) Applicant: BENEVOLENTAI TECHNOLOGY LIMITED, London (GB)

(72) Inventors: Dean PLUMBLEY, London (GB); Matthew SELLWOOD, London (GB); Marco FISCATO, London (GB); Alain Claude VAUCHER, London (GB)

(73) Assignee: BENEVOLENTAI TECHNOLOGY LIMITED, London (GB)

(21) Appl. No.: 17/041,528

(22) PCT Filed: Mar. 29, 2019
(86) PCT No.: PCT/GB2019/050923

§ 371 Date: Sep. 25, 2020

(30) Foreign Application Priority Data
Mar. 29, 2018 (GB) 1805302.5

(51) Int. Cl. G06N 20/20; G06K 9/62

(52) U.S. Cl. CPC G06N 20/20; G06K 9/6262; G06K 9/6261; G06K 9/6227; G06K 9/6288; G06K 9/6202

(57) ABSTRACT

Method(s), apparatus and system(s) are provided for generating and using an ensemble model. The ensemble may be generated by training a plurality of models based on a plurality of datasets associated with compounds; calculating model performance statistics for each of the plurality of trained models; selecting and storing a set of optimal trained model(s) from the trained models based on the calculated model performance statistics; and forming one or more ensemble models, each ensemble model comprising multiple models from the set of optimal trained model(s). The ensemble model may be used by retrieving the ensemble model and inputting, to the ensemble model, data representative of one or more labelled dataset(s) used to generate and/or train the model(s) of the ensemble model; and receiving, from the ensemble model, output data associated with labels of the one or more labelled dataset(s).

The present application relates to a system and method for ensemble model creation and selection.

### BACKGROUND

Informatics is the application of computer and informational techniques and resources for interpreting data in one or more academic and/or scientific fields. Cheminformatics' (also known as chem(o)informatics) and bioinformatics may be the application of computer and informational techniques and resources for interpreting chemical and/or biological data. This may include solving and/or modelling processes and/or problems in the field(s) of chemistry and/or biology. For example, these computing and information techniques and resources may transform data into information, and subsequently information into knowledge for rapidly making improved decisions in, by way of example only but not limited to, the field of drug lead identification, discovery and optimisation.

Machine learning techniques are computational methods that can be used to devise complex analytical models and algorithms that lend themselves to solving complex problems such as prediction and analysis of complex processes. The analytical models may learn from historical relationships and trends in the associated data and allow researchers, data scientists, engineers, and analysts to make rapid and improved decisions and/or uncover hidden insights. ML techniques can be used to generate analytical models in the drug discovery, identification, and optimization and other related cheminformatics and/or bioinformatics fields. The analytical models may solve problems, model processes and/or form predictions in relation to, by way of example only but not limited to, compound interactions with other molecules (e.g. proteins, deoxyribonucleic acid (DNA), ribonucleic acid (RNA), etc. . . . ) or other compounds, physiochemical properties of compounds, solvation properties of compounds, drug properties of compounds, structures and/or material properties of compounds, or any other suitable process and/or prediction associated with molecules and/or compounds and the like etc.

There are a myriad of ML techniques that may be selected for generating models of chemical or biological problems/processes of interest that may assist in, by way of example only but is not limited to, the prediction of compounds and/or drugs in drug discovery. Most researchers, data scientists and engineers use a trial and error approach when applying ML techniques to generate models for solving various problems in cheminformatics and/or bioinformatics. For example, each of the different ML techniques used to generate each model needs to be initially configured to operate optimally for training and generating a trained model for modelling a particular problem/process. The initial configuration uses so-called hyperparameter(s), which are parameter values used by a chosen ML technique for generating a model and cannot be estimated from the training data but, instead, need to be selected a priori for a given ML technique and predictive modelling problem/process. The time required to train and test a ML technique to generate a model can greatly depend upon the choice of its hyperparameters. The best hyperparameter values to use for a given modelling problem/process is typically unknown to the researcher or data scientist. The selection of the hyperparameters for each ML technique to generate a model is commonly based on user experience, rules of thumb, copying hyperparameter values used in other problems/processes or models, or by trial and error.

Furthermore, most researchers and/or data scientists do not fully appreciate or understand how changing hyperparameters, selection of ML technique from the myriad of ML techniques, and/or type of input data format can affect the output of a model such as, by way of example only but not limited to, the predictive capabilities and/or modelling accuracy of the resulting model. Conventionally, researchers have been found to use default hyperparameters and any type of input data format rather than going to the time and trouble to find the most optimal solution for modelling a particular problem or process. For example, for a model based on a random forest (RF) ML technique, having too many RF trees may lead to the danger of overfitting whereas too few RF trees may lead to reduced accuracy. It has been found that the number of RF trees depends on training dataset size and/or format.

Other factors that greatly affect predictive ability and/or modelling accuracy when generating a model to solve cheminformatics and/or bioinformatics problems/processes include, by way of example only but is not limited to, the selection of the ML technique for the model, the formatting and style of input data, and the amount of labelled datasets for training the model. Thus, the researcher/data scientist or operator is faced with a multi-faceted optimisation problem when generating a model for cheminformatics/bioinformatics problem/processes that can be unrealistic to solve manually using user experience, rules of thumb, copying hyperparameter values used in other problems or models, or by trial and error in which the result is most likely an ill-fitted or sub-optimal model.

There is a desire to improve the modelling of cheminformatics/bioinformatics problems, to improve the selection of ML techniques and make improved models that are more accurate and can make full use of the available cheminformatics and/or bioinformatics datasets. There is also a desire to avoid or reduce operator error in, by way of example only but not limited to, selecting the wrong model, wrong hyperparameters for a model, incompatible dataset format, and, in turn, reducing the likelihood of incorrect decision making and associated costs based on poor model predictions and/or accuracy.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides a method(s), apparatus and/or system(s) for modelling a process or problem associated with compound(s) by inputting, to an ensemble model for modelling the process or problem, representations of one or more compound(s); receiving, from the ensemble model, results associated with modelling the process or problem based on the one or more compound(s). The ensemble model includes multiple model(s) automatically selected based on model performance statistics calculated for each of the model(s).

For example, the multiple model(s) of the ensemble model may be selected from a subset of the best performing trained models that have been optimised for modelling the process or problem associated with one or more compounds. The subset of the best performing trained models are determined based on model performance statistics of a plurality of trained models. Each of the trained models may be trained based on one or more ML technique(s) or a plurality of ML technique(s), a corresponding plurality of sets of hyperparameters, one or more labelled datasets and/or dataset folds associated with compounds. Each labelled dataset and corresponding dataset folds may be duplicated multiple times, with each duplicate being modified based on a different compound descriptor format from a plurality of compound descriptor formats. The trained models may be assessed based on model performance statistics of the models and the best performing trained models selected and stored for forming the one or more ensemble model(s).

In a first aspect, the present disclosure provides a computer-implemented method of generating an ensemble model, the method comprising: training a plurality of models based on a plurality of datasets associated with compounds; calculating model performance statistics for each of the plurality of trained models; selecting and storing a set of optimal trained model(s) from the trained models based on the calculated model performance statistics; and forming one or more ensemble models, each ensemble model comprising multiple models from the set of optimal trained model(s).

Preferably, calculating model performance statistics further comprises cross-validating each of the plurality of models.

Preferably, calculating the model performance statistics for each trained model comprises calculating at least one or more model performance statistics for each trained model based on one or more from the group of: positive predictive value or precision of the trained model; sensitivity, specificity, true predictive rate, or recall of the trained model; a receiver operating characteristic, ROC, graph associated with the trained model; an area under a ROC curve associated with the trained model; an area under a precision ROC curve associated with the trained model; an area under a precision and recall ROC curve associated with the trained model; F1 score; r-squared; root mean squared error; mean squared error; median absolute error; mean absolute error; any other function associated with precision and/or recall of the trained model; and any other model performance statistic(s) for evaluating each of the trained models based on model type or machine learning technique associated with each model.

Preferably, the method further comprises: generating a plurality of datasets from a set of labelled datasets associated with compounds.

Preferably, generating the plurality of datasets further comprises generating groups of datasets from the set of labelled datasets based on a plurality of compound descriptors, wherein each group of datasets corresponds to a different compound descriptor.

Preferably, a compound descriptor comprises a compound descriptor based on at least one or more of: International Chemical Identifier, InChI; InChIKey; MoIFile format; two dimensional Physical Chemical descriptors; three dimensional Physical Chemical descriptors; XYZ file format; Extended Connectivity Fingerprint, ECFP; Structure Data Format; structural formula or representation of the compound; Simplified Molecular Input Line Entry Specification, SMILES, strings or format; SMILES arbitrary target specification or format; Chemical Mark-up Language format; and any other chemical descriptor or chemical descriptor format for describing, representing and/or encoding molecular information and/or structure(s) of compounds.

Preferably, generating the plurality of datasets further comprising generating, for each dataset of the plurality of datasets, a set of dataset folds by partitioning said each dataset into multiple portions; and for the plurality of models and the plurality of datasets, performing the steps of: training each model based the set of dataset folds corresponding to each dataset; calculating model performance statistics for each trained model based on each fold of the set of dataset folds corresponding to each dataset; and storing data representative of the trained model in a set of optimal models based on the calculated model performance statistics.

Preferably, storing data representative of the trained model further comprises storing data representative of the trained model in the set of optimal models by comparing the calculated model statistics with one or more performance thresholds associated with the model statistics.

Preferably, storing data representative of the trained model further comprises storing data representative of the trained model in the set of optimal models by comparing the calculated model statistics with the calculated model statistics of previously stored models.

Preferably, the method further comprising deleting previously stored models from the set of optimal models based on the calculated model statistics of a model of the same type.

Preferably, storing data representative of the trained model further comprises storing data representative of the trained model, the calculated model statistics of the trained model, and/or the dataset associated with training the trained model.

Preferably, the method further comprising repeating the steps of training, calculation and storing for each of a set of hyperparameters selected from a plurality of hyperparameters associated with said each model.

Preferably, the plurality of models further comprises models configured based on a set hyperparameters selected from a plurality of hyperparameters associated with each type of model of the plurality of models.

Preferably, forming one or more ensemble of models further comprises selecting a subset of optimal models from the set of optimal model(s), wherein each model in the subset of optimal models has improved model statistics compared with the remaining models in the set of optimal models.

Preferably, selecting a subset of optimal models from the set of optimal model(s) further comprises ranking the optimal models based on the model statistics and selecting a subset of the topmost ranked optimal models for inclusion into the ensemble model.

Preferably, selecting a subset of optimal models from the set of optimal model(s), further comprises: retrieving models and associated model statistics from the set of optimal models that correspond to the same model type; ranking the retrieved models based on the model statistics; and selecting one or more model(s) from the retrieved models having the highest model statistics for inclusion into the ensemble model.

Preferably, selecting a subset of optimal models from the set of optimal model(s), further comprises, for each of the plurality of datasets: retrieving the models and associated model statistics from the set of optimal models that are associated with the same dataset; ranking the retrieved models based on the model statistics; and selecting one or more topmost model(s) from the ranked retrieved models for inclusion into the ensemble model.

Preferably, the method further comprising benchmarking the one or more ensemble models based on the plurality of datasets.

Preferably, benchmarking the one or more ensemble models further comprises calculating ensemble model statistics based on cross-validating each of the one or more ensemble models.

Preferably, the computer-implemented method further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

In a second aspect, the present disclosure provides a computer-implemented method for using an ensemble model, wherein the ensemble model is based on an ensemble model generated according to according to the first aspect, modifications thereof and/or as described herein, the method comprising: inputting, to the ensemble model, data representative of one or more labelled dataset(s) used to generate and/or train the model(s) of the ensemble model; and receiving, from the ensemble model, output data associated with labels of the one or more labelled dataset(s).

Preferably, the computer-implemented method further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

In a third aspect, the present disclosure provides a computer-implemented method for modelling a process or problem associated with compound(s), the method comprising: inputting, to an ensemble model for modelling the process or problem, representations of one or more compound(s); receiving, from the ensemble model, results associated with modelling the process or problem based on the one or more compound(s); and wherein the ensemble model comprises multiple model(s) automatically selected based on model performance statistics calculated for each of the model(s).

Preferably, the computer-implemented method further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

In a fourth aspect, the present disclosure provides an apparatus comprising a processor, a memory unit and a communication interface, wherein the processor is connected to the memory unit and the communication interface, wherein the processor and memory are configured to implement the computer-implemented method according to the first aspect, modifications thereof and/or as described herein.

In a fifth aspect, the present disclosure provides an ensemble model comprising data representative of a set of models generated according to the first aspect, modifications thereof and/or as described herein.

In a sixth aspect, the present disclosure provides an ensemble model obtained by the computer-implemented method according to the first aspect, modifications thereof and/or as described herein.

In a seventh aspect, the present disclosure provides a computer-readable medium comprising data or instruction code representative of an ensemble model according to any one of the fifth or sixth aspects, modifications thereof and/or as described herein, which when executed on a processor, causes the processor to implement the ensemble model.

In a eighth aspect, the present disclosure provides a computer-readable medium comprising data or instruction code, which when executed on a processor, causes the processor to implement the computer-implemented method according to the first aspect, modifications thereof and/or as described herein.

In a ninth aspect, the present disclosure provides a computer-readable medium comprising data or instruction code, which when executed on a processor, causes the processor to implement the computer-implemented method according to the second aspect, modifications thereof, and/or as described herein.

In a tenth aspect, the present disclosure provides a computer-readable medium comprising data or instruction code, which when executed on a processor, causes the processor to implement the computer-implemented method according to the third aspect, modifications thereof, and/or as described herein.

In an eleventh aspect, the present disclosure provides a tangible (or non-transitory) computer-readable medium comprising data or instruction code, which when executed on one or more processor(s), causes at least one of the one or more processor(s) to perform at least one of the steps of the method of: training a plurality of models based on the plurality of datasets associated with compounds; calculating model performance statistics for each of the plurality of trained models; selecting and storing a set of optimal trained model(s) from the trained models based on the calculated model performance statistics; and forming one or more ensemble models, each ensemble model comprising multiple models from the set of optimal trained model(s).

Preferably, the computer-readable medium further comprising data or instruction code, which when executed on a processor, causes the processor to implement one or more steps of the computer-implemented method according to the first aspect, modifications thereof, and/or as described herein.

In an twelfth aspect, the present disclosure provides an apparatus comprising a processor and a memory unit, the processor is connected to the memory unit, wherein: the processor is configured to train a plurality of models based on a plurality of datasets associated with compounds; the processor is configured to calculate model performance statistics for each of the plurality of trained models; the processor and memory are configured to selecting and storing a set of optimal trained model(s) from the trained models based on the calculated model performance statistics; and the processor and memory are configured to form one or more ensemble models, each ensemble model comprising multiple models from the set of optimal trained model(s).

Preferably, the apparatus further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

In a thirteenth aspect, the present disclosure provides an apparatus comprising a processor, a memory unit and a communication interface, the processor is connected to the memory unit and the communication interface, wherein: the processor and communication interface are configured to retrieve an ensemble model generated according to any one of the first, eleventh, or twelfth aspects, modifications thereof and/or as described herein, in which the processor and memory are configured to input, to the ensemble model, data representative of one or more labelled dataset(s) used to generate and/or train the model(s) of the ensemble model; and the processor and memory are configured to receive, from the ensemble model, output data associated with labels of the one or more labelled dataset(s).

Preferably, the apparatus further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

In a fourteenth aspect, the present disclosure provides an apparatus comprising a processor, a memory unit and a communication interface, the processor is connected to the memory unit and the communication interface, wherein: the processor is configured to input, to an ensemble model for modelling a process or problem associated with compounds, representations of one or more compound(s); the processor and memory are configured to receive, from the ensemble model, results associated with modelling the process or problem based on the one or more compound(s); and wherein the ensemble model comprises multiple model(s) automatically selected based on model performance statistics calculated for each of the model(s).

Preferably, the apparatus further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

In a fifteenth aspect, the present disclosure provides a system for generating an ensemble model, the system comprising: a dataset generation module configured for generating a plurality of datasets associated with compounds based on multiple labelled datasets; a model generation module configured to train a plurality of models based on the plurality of datasets associated with compounds, wherein model performance statistics are calculated for each of the plurality of trained models; a model selection module configured to select and store a set of optimal trained model(s) from the plurality of trained models based on the calculated model performance statistics; and a ensemble creation module configured to retrieve multiple models from the set of optimal trained models and form one or more ensemble models, each ensemble model comprising multiple models from the set of optimal trained model(s).

Preferably, the system further comprising: an ensemble benchmark module configured to retrieve a formed ensemble model and benchmark the retrieved ensemble model based on the corresponding plurality of datasets used to generate each of the models forming the ensemble model; and an ensemble database module configured to store the benchmarked ensemble models and benchmark results.

Preferably, the system is further configured to implement the computer-implemented method according to any of the first, eleventh, and twelfth aspects, modifications thereof, and/or as described herein.

Preferably, the system further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

Preferably, the computer-implemented method, apparatus or system according to any one of the first to fifteenth aspects, combinations and/or modifications thereof, and/or as described herein, wherein training the plurality of models further comprises splitting the ensemble generation into a plurality of model training tasks or jobs, wherein each model training task is associated with a model of the plurality of models and a dataset of the plurality of datasets associated with compounds; and submitting each model training task or job to a plurality of servers for training the model associated with said each model training task or job.

Preferably, the computer-implemented method, apparatus or system according to any one of the first to fifteenth aspects, combinations and/or modifications thereof, and/or as described herein, wherein each of the model training tasks or jobs calculate model performance statistics for the associated trained model, and, receiving from each of the plurality of model training tasks or jobs, the calculated model performance statistics for selecting and storing a set of optimal trained model(s) from the trained models based on the calculated model performance statistics of each trained model.

Preferably, the computer-implemented method, apparatus or system according to any one of the first to fifteenth aspects, combinations and/or modifications thereof, and/or as described herein, further comprising storing each trained model of the set of optimal trained models in a model file object or model file including data representative of at least one or more from the group of: the trained model, hyperparameters associated with the trained model, chemical or compound descriptor associated with the trained model, dataset used for training the trained model, and model performance statistics.

Preferably, the computer-implemented method, apparatus or system according to any one of the first to fifteenth aspects, combinations and/or modifications thereof, and/or as described herein, further comprising storing each ensemble model formed from multiple models of the set of optimal trained model(s) in a ensemble model file object or ensemble model file including data representative of at least one from the group of: the multiple models, the file objects associated with the multiple models, datasets used for training the multiple models, hyperparameters associated with each of the multiple models, model performance statistics of the ensemble model and/or multiple models.

Preferably, the computer-implemented method, apparatus or system according to any one of the first to fifteenth aspects, combinations and/or modifications thereof, and/or as described herein, wherein each ensemble training task or job further includes a set of hyperparameters associated with the model.

The methods described herein may be performed by software in machine readable form on a tangible (or non-transitory) storage medium or tangible computer-readable medium e.g. in the form of a computer program comprising computer program code means adapted to perform all the steps of any of the methods described herein when the program is run on a computer and where the computer program may be embodied on a computer readable medium. Examples of tangible (or non-transitory) storage media or computer-readable media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a flow diagram illustrating an example system for generating an ensemble model according to the invention;

FIG. 1b is a flow diagram illustrating an example system for using an ensemble model according to the invention;

FIGS. 2a-2g are schematic diagrams illustrating an example apparatus for generating an ensemble model according to the invention;

FIG. 3 is a diagram illustrating the complexity of generating an ensemble model according to the invention;

FIG. 4a is a schematic diagram of a computing device according to the invention;

FIG. 4b is a schematic diagram of a system according to the invention;

FIG. 5a is a schematic diagram of an example system for generating an ensemble model ensemble model according to the invention;

FIG. 5b is a schematic diagram of another example system for generating an ensemble model according to the invention;

FIG. 5c is a schematic diagram of an example file storage system and model files for storing one or more models from example systems of FIG. 5a and/or 5b according to the invention; and

FIG. 5d is a schematic diagram of an example model report file or file object according to the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

It has been recognised that most researchers and/or data scientists do not fully appreciate or understand how changing hyperparameters, selection of ML technique, and/or type of input data format can affect the predictive capabilities and/or modelling accuracy of a model based on a ML technique, let alone an ensemble model based on one or more ML technique(s). This yields a multi-faceted optimisation problem for modelling a cheminformatics and/or bioinformatics problem or process that is unrealistic to solve manually using user experience, rules of thumb, copying hyperparameter values used in other problems or models, or by trial and error.

The inventors have advantageously developed a system for generating and selecting from a large number of trained models, or a plurality of sets of trained models, with the same or similar objectives a subset of the best performing trained models that can be used to create one or more ensemble model(s) that have been optimised for modelling a process or problem associated with one or more compounds. The trained models are based on one or more ML technique(s) or a plurality of ML technique(s) and corresponding plurality of sets of hyperparameters, one or more labelled datasets and/or dataset folds associated with compounds. The trained models are assessed based on model performance statistics (MPSs) of the models and the best performing trained models selected and stored for forming the one or more ensemble model(s).

A compound may comprise or represent a chemical or biological substance composed of one or more molecules (or molecular entities), which are composed of atoms from one or more chemical element(s) (or more than one chemical element) held together by chemical bonds. Example compounds as used herein may include, by way of example only but are not limited to, molecules held together by covalent bonds, ionic compounds held together by ionic bonds, intermetallic compounds held together by metallic bonds, certain complexes held together by coordinate covalent bonds, drug compounds, biological compounds, biomolecules, biochemistry compounds, one or more proteins or protein compounds, one or more amino acids, lipids or lipid compounds, carbohydrates or complex carbohydrates, nucleic acids, deoxyribonucleic acid (DNA), DNA molecules, ribonucleic acid (RNA), RNA molecules, and/or any other organisation or structure of molecules or molecular entities composed of atoms from one or more chemical element(s) and combinations thereof.

ML technique(s) are used to train and generate one or more trained models having the same or a similar output objective associated with compounds. ML technique(s) may comprise or represent one or more or a combination of computational methods that can be used to generate analytical models and algorithms that lend themselves to solving complex problems such as, by way of example only but is not limited to, prediction and analysis of complex processes and/or compounds. ML techniques can be used to generate analytical models associated with compounds for use in the drug discovery, identification, and optimization and other related informatics, cheminformatics and/or bioinformatics fields.

Examples of ML technique(s) that may be used by the invention as described herein may include or be based on, by way of example only but is not limited to, any ML technique or algorithm/method that can be trained on a labelled and/or unlabelled datasets to generate a model associated with the labelled and/or unlabelled dataset, one or more supervised ML techniques, semi-supervised ML techniques, unsupervised ML techniques, linear and/or non-linear ML techniques, ML techniques associated with classification, ML techniques associated with regression and the like and/or combinations thereof. Some examples of ML techniques may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), deep NNs, deep learning, deep learning ANNs, inductive logic programming, support vector machines (SVMs), sparse dictionary learning, clustering, Bayesian networks, reinforcement learning, representation learning, similarity and metric learning, sparse dictionary learning, genetic algorithms, rule-based machine learning, learning classifier systems, and/or one or more combinations thereof and the like.

Some examples of supervised ML techniques may include or be based on, by way of example only but is not limited to, ANNs, DNNs, association rule learning algorithms, a priori algorithm, Éclat algorithm, case-based reasoning, Gaussian process regression, gene expression programming, group method of data handling (GMDH), inductive logic programming, instance-based learning, lazy learning, learning automata, learning vector quantization, logistic model tree, minimum message length (decision trees, decision graphs, etc.), nearest neighbour algorithm, analogical modelling, probably approximately correct learning (PAC) learning, ripple down rules, a knowledge acquisition methodology, symbolic machine learning algorithms, support vector machines, random forests, ensembles of classifiers, bootstrap aggregating (BAGGING), boosting (meta-algorithm), ordinal classification, information fuzzy networks (IFN), conditional random field, anova, quadratic classifiers, k-nearest neighbour, boosting, sprint, Bayesian networks, Nave Bayes, hidden Markov models (HMMs), hierarchical hidden Markov model (HHMM), and any other ML technique or ML task capable of inferring a function or generating a model from labelled training data and the like.

Some examples of unsupervised ML techniques may include or be based on, by way of example only but is not limited to, expectation-maximization (EM) algorithm, vector quantization, generative topographic map, information bottleneck (IB) method and any other ML technique or ML task capable of inferring a function to describe hidden structure and/or generate a model from unlabelled data and/or by ignoring labels in labelled training datasets and the like. Some examples of semi-supervised ML techniques may include or be based on, by way of example only but is not limited to, one or more of active learning, generative models, low-density separation, graph-based methods, co-training, transduction or any other a ML technique, task, or class of supervised ML technique capable of making use of unlabeled datasets and labelled datasets for training (e.g. typically the training dataset may include a small amount of labelled training data combined with a large amount of unlabeled data and the like.

Some examples of artificial NN (ANN) ML techniques may include or be based on, by way of example only but is not limited to, one or more of artificial NNs, feedforward NNs, recursive NNs (RNNs), Convolutional NNs (CNNs), autoencoder NNs, extreme learning machines, logic learning machines, self-organizing maps, and other ANN ML technique or connectionist system/computing systems inspired by the biological neural networks that constitute animal brains and capable of learning or generating a model based on labelled and/or unlabelled datasets. Some examples of deep learning ML technique may include or be based on, by way of example only but is not limited to, one or more of deep belief networks, deep Boltzmann machines, DNNs, deep CNNs, deep RNNs, hierarchical temporal memory, deep Boltzmann machine (DBM), stacked Auto-Encoders, and/or any other ML technique capable of learning or generating a model based on learning data representations from labelled and/or unlabelled datasets.

It is to be appreciated that there are a myriad of ML techniques that may be used to train and generate a plurality of trained models, in which each trained model is associated with the same or a similar output objective in relation to compounds. Each of the different ML techniques used to train and generate each trained model needs to be initially configured to operate optimally for training and generating the trained model for modelling a particular problem/process associated with compounds. The initial configuration uses so-called hyperparameter(s). Hyperparameters for a particular ML technique may comprise or represent one or more or a plurality of parameter values that are initially used to configure the particular ML technique when training and generating a trained model. Hyperparameters may have parameter values that are, by way of example only but is not limited to, at least one of one or more continuous values, one or more integer values, one or more conditional values or textual values representing one of a selection of functions an ML technique may use. Furthermore, the existence of some hyperparameters is conditional upon the value of others (e.g. the size of each hidden layer in a neural network can be conditional upon the number of layers). The parameter values of the hyperparameters are selected a priori for a given ML technique and can affect not only the training and generation of the trained model modelling, by way of example only but not limited to, a complex problem or process (e.g. a predictive modelling problem/process) but also the trained model's performance such as prediction accuracy after training. A trained model's performance may be measured by model performance statistics (MPSs) such as, by way of example only but not limited to, statistics associated with prediction and/or recall accuracy and the like.

Each of trained model may comprise or represent data representative of an analytical model that is associated with modelling a particular process, problem and/or prediction associated with compounds in the informatics, cheminformatics and/or bioinformatics fields. An ensemble model may comprise or represent data representative of multiple trained models (e.g. two or more) that are associated with the same or a similar output objective and/or associated with modelling the same or similar process, problem and/or prediction associated with compounds in the informatics, cheminformatics, and/or bioinformatics fields. An ensemble model may be generated by selecting multiple trained models from a plurality of trained models, where each of the trained models in the plurality of trained models are associated with the same or a similar output objective and/or associated with modelling the same or similar process, problem and/or prediction associated with compounds.

Examples of output objective(s) and/or modelling a process, problem and/or prediction associated with compounds in the informatics, cheminformatics, and/or bioinformatics fields may include one or more of, by way of example only but is not limited to, compound interactions with other compounds and/or proteins, physiochemical properties of compounds, solvation properties of compounds, drug properties of compounds, structures and/or material properties of compounds and the like etc., and/or modelling chemical or biological problems/processes/predictions of interest that may assist in, by way of example only but is not limited to, the prediction of compounds and/or drugs in drug discovery, identification and/or optimisation.

Other examples of output objectives and/or modelling a process, problem and/or prediction associated with compounds may include, by way of example only but is not limited to, modelling or predicting a characteristic and/or property of compounds, modelling and/or predicting whether a compound has a particular property, modelling or predicting whether a compound binds to, by way of example only but is not limited to, a particular protein, modelling or predicting whether a compound docks with another compound to form a stable complex, modelling or predicting whether a particular property is associated with a compound docking with another compound (e.g. ligand docking with a target protein); modelling and/or predicting whether a compound docks or binds with one or more target proteins; modelling or predicting whether a compound has a particular solubility or range of solubilities, or any other property.

Further examples of output objectives and/or modelling a process, problem and/or prediction associated with compounds, may include, by way of example only but is not limited to, outputting, modelling and/or predicting physiochemical properties of compounds such as, by way of example only but not limited to, one or more of Log P, pKa, freezing point, boiling point, melting point, polar surface area or any other physiochemical property of interest in relation to compounds; outputting, modelling and/or predicting solvation properties of compounds such as, by way of example only but not limited to, phase partitioning, solubility, colligative properties or any other properties of interest in relation to compounds; modelling and/or predicting one or more drug properties of compounds such as, by way of example only but not limited to, dosage, dosage regime, binding affinity, adsorption (e.g. gut, cellular etc.), metabolism, brain penetrance, toxicity and any other drug property of interest in relation to compounds; outputting, modelling and/or predicting binding modes of compounds such as, by way of example only but not limited to, one or more of predictive co-crystal structures of ligands to receptors and the like; outputting, modelling and/or predicting crystal structures of compounds such as, by way of example only but not limited to, one or more of crystal packing of compounds, protein folding, and any other crystal structure type and the like that may be of interest in relation to compounds; outputting, modelling and/or predicting materials properties of compounds such as, by way of example only but not limited to, one or more of conductivity, surface tension, coefficient of friction, permeability, hardness, tensile strength, luminosity etc., and any other material property that may be of interest in relation to compounds; outputting, modelling and/or predicting any other properties of interest, interactions of interest, characteristics of interest, or anything else of interest in relation to compounds in the informatics, cheminformatics and/or bioinformatics fields.

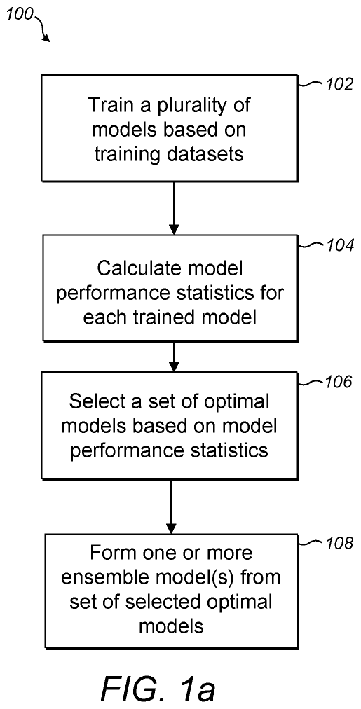

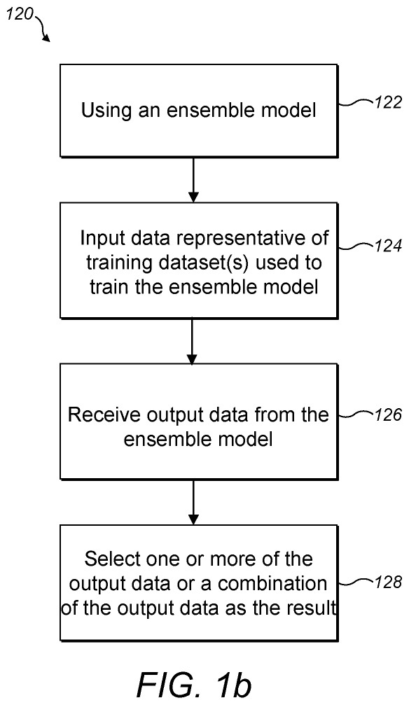

FIG. 1a is a flow diagram illustrating an example ensemble generation process 100 for generating an ensemble model according to the invention. The ensemble model may comprise or represent multiple trained models that are directed to have the same output objective and/or capable of modelling the same or similar process, problem or prediction associated with compounds. The steps of the process may include one or more of the following steps: In step 102, a plurality of models are trained based on a plurality of datasets associated with compounds. The plurality of models are trained based on the same output objective or configured for modelling the same or similar process, problem or prediction associated with compounds. For example, the plurality of datasets may include a plurality of labelled datasets associated with compounds. The plurality of models may be based on a set of machine learning (ML) techniques. The plurality of models may include multiple groups of models in which the models in each group of models correspond to a particular type of ML technique or model type. Each of the plurality of models are trained on each of the plurality of datasets forming a plurality of trained models. Once one or more models have been trained or the plurality of the models have been trained, the process 100 may proceed to step 104.

In step 104, each trained model is assessed and MPSs are calculated for each trained model of the plurality of trained models. The MPSs may include any MPS that is representative of the performance of the trained model on the labelled dataset(s) and/or unlabelled dataset(s) associated with the trained model. In step 106, the MPSs for each trained model are analysed and used to select and/or store a set of “optimal” trained model(s) from the trained models. The set of optimal trained model(s) are optimal in the sense that the trained models that are selected have the most improved MPSs over the plurality of trained models. Once a set of optimal trained models has been generated or selected, in step 108, one or more ensemble models may be formed or selected, in which each ensemble model comprises multiple trained models selected from the set of optimal trained model(s).

As described, step 102 may include retrieving, using and/or generating a plurality of datasets for training the plurality of models. The plurality of datasets may include a plurality of labelled datasets associated with compounds. The ensemble generation process 100 may further generate, use and/or retrieve suitable labelled datasets for training the plurality of models. There may be a plurality of chemical or compound descriptors or chemical/compound input formats, hereinafter referred to as CDs. For example, each labelled dataset may be used to generate a set of chemical or compound descriptor (CD) labelled datasets based on one or more selected CDs or a plurality of CDs for inclusion into the plurality of datasets. Each set of CD labelled datasets includes the same labelled dataset but described by a different CD from the plurality of CDs. This may be achieved by replicating each labelled dataset based on the number of plurality of CDs, and then modifying the compounds described in each replicated labelled dataset to be based on a different CD or compound input format selected from a plurality of CDs. As another example, the plurality of datasets may be generated from the set of labelled datasets in which groups of CD labelled datasets for each labelled dataset in the set of labelled datasets are generated based on a plurality of CDs, where each CD is different.

Furthermore, the set of ML techniques may include, but way of example only but is not limited to, random forests, state vector machines, linear ML techniques, XGBoost, neural networks, and any other ML technique suitable for use in modelling processes and/or problems associated with compounds. The plurality of models may include multiple groups of models, where the models in each group of models correspond to a particular type of ML technique or model type. The models in each group may be of the same model type but may differ based on the selection of hyperparameters used to configure each model and/or based on the labelled dataset used to train that model. The hyperparameters for each model may be selected from a plurality of hyperparameters associated with that model type. Each of the plurality of models are trained on each of the plurality of datasets forming a plurality of trained models.

Step 104 may further include calculating the MPSs using cross-fold-validation for each of the plurality of models. Cross-validating each of the plurality of models may require generating multiple folds for each dataset of the plurality of datasets, training said each model on each of the multiple folds to generate a MPS, and combining the MPSs from each fold to generate a combined MPS for that model and that dataset. The MPSs of a trained model may comprise or represent an indication or a measure of the accuracy and/or performance of the trained model. The MPSs for each trained model may be based on, by way of example but is not limited to, one or more from the group of: positive predictive value or precision of the trained model; sensitivity, true predictive rate, or recall of the trained model; a receiver operating characteristic, ROC, graph associated with the trained model; an area under a ROC curve associated with the trained model (e.g. AUC); an area under a precision and/or recall ROC curve (e.g. AUpC and/or AUprC) associated with the trained model; any other function associated with precision and/or recall of the trained model; and any other MPS(s) for evaluating each of the trained models.

MPSs may be based on the category of ML technique used. For example, if the ML technique used to train and generate a trained model is classification based, then the MPSs that may be used may include or be based on, by way of example only but is not limited to, area under the curve (AUC), area under the precision recall curve (AUprC), F1 score, precision, recall, accuracy, sensitivity, and/or specificity and the like. If the ML technique used to train and generate a trained model is regression based, then the MPSs that may be used may include or be based on, by way of example only but is not limited to, r2 (r squared), root mean squared error (RMSE), mean squared error (MSE), median absolute error, mean absolute error and the like. It is to be appreciated that for any other category of ML technique used to train and generate a trained model, then the MPS that may be used may be based on one or more of the suitable MPSs associated with assessing, by way of example only but is not limited to, the performance and/or accuracy of the trained model based each type of model such as the category of ML technique used to generate the model.

The ensemble generation process 100 may further include one or more steps such as stacking each ensemble model using a combiner ML technique or algorithm to generate, based on the labelled training datasets of the models of the ensemble model, a combiner ML model for combining the predictions or outputs of each of the models to form a final prediction or data output representing the output of the ensemble model.

The ensemble generation process 100 may be implemented by an apparatus, computing device or system that may include, by way of example only but is not limited to, a processor, a memory unit and/or a communication interface. The processor may be connected to the memory unit and/or the communication interface. The processor, memory and/or communication interface may be configured to implement the ensemble generation process 100. For example, the processor may be configured to train a plurality of models based on a plurality of datasets associated with compounds. The processor may be further configured to calculate model performance statistics for each of the plurality of trained models. The processor and memory may be further configured to select and store a set of optimal trained model(s) from the trained models based on the calculated model performance statistics. The processor, memory and/or communication interface may be configured to form one or more ensemble models, each ensemble model comprising multiple models from the set of optimal trained model(s); and store the one or more ensemble models in an ensemble model database and the like. The apparatus may be further configured to implement the ensemble generation process 100 and/or functionality of apparatus, systems, method(s) and/or process(es) as described herein and/or as described with reference to FIGS. 1a-4b.

FIG. 1b is a flow diagram illustrating an example process 120 for using an ensemble model according to the invention. The ensemble model may be configured for modelling a process or problem associated with compound(s). The ensemble model may include multiple trained model(s) automatically selected based on MPSs calculated for each of the trained model(s). The multiple models may be, by way of example only but is not limited to, selected from a set of optimal trained models as generated by process 100, in which the selected multiple models may be combined to form an ensemble model. The steps of the process 120 may include one or more of the following steps:

In step 122, an ensemble model may be selected from a set of ensemble models for use in modelling the process or a problem associated with compounds. The ensemble model may be based on multiple models selected from a set of optimal trained models. Additionally or alternatively, the ensemble model may be selected and retrieved from a set of ensemble models that have been previously assessed/benchmarked and stored. In step 124, the selected ensemble model includes multiple trained models, the input data may comprise data representative of one or more representation(s) of one or more compound(s). For example, the input data may be representative of the compounds associated with, the same and/or most like different or dissimilar to, the compounds used in the training datasets for generating or training each model. This input data for each model may be input to the ensemble model. The input data is tailored or formatted in a form suitable for input to each trained model in the ensemble model. Thus multiple forms of input data will be input to the ensemble model, each form for the corresponding model of the ensemble model. For example, each model may accept input data associated with compounds based on one of a plurality of chemical or compound descriptors. Once input, each of the models in the ensemble model are configured to process the corresponding input data and output result data accordingly. In step 126, output result data may be received from the ensemble model. The output result data may be correspond to each of the output data from each of the models in the ensemble model. The output data from each model may be associated with the labels of labelled training data used to train the corresponding model of the ensemble model. Alternatively or additionally, the output result data may be a weighted combination of the output data from each of the models of the ensemble model. The results from the ensemble model are associated with modelling the process or problem based on the one or more compound(s).

The example process 120 may be implemented by an example apparatus that may include, by way of example only but is not limited to, a processor, a memory unit and a communication interface, the processor is connected to the memory unit and the communication interface. For example, the processor and communication interface may be configured to retrieve an ensemble model generated according to the ensemble generation process 100 and/or as described herein and/or as described with reference to any of FIGS. 1a to 4b. That is, the apparatus for implementing example process 120 may retrieve an ensemble model that is applicable for use with the input dataset from the ensemble model database. The processor and memory may be further configured to input, to the ensemble model, data representative of one or more compounds and/or data suitable for inputting to the ensemble model, the model(s) of which were trained based on one or more labelled dataset(s). The data representative of the one or more compounds may be suitable for the model(s) to model a process or problem associated with compounds. The processor and memory may also be further configured to receive, from the ensemble model, output data associated with labels of the one or more labelled dataset(s).

Another example apparatus may include, by way of example only but is not limited to, a processor, a memory unit and a communication interface. The processor is connected to the memory unit and the communication interface. The processor may be configured to input, to an ensemble model for modelling a process or problem associated with compounds, representations of one or more compound(s). The processor and memory may be further configured to receive, from the ensemble model, results associated with modelling the process or problem based on the one or more compound(s). The ensemble model includes multiple model(s) automatically selected based on model performance statistics calculated for each of the model(s). For example, the ensemble model may be generated based on ensemble model generation process 100 as described with reference to FIG. 1a, and/or based on the apparatus, systems, method(s) and/or process(es) as described herein or as described with reference to FIGS. 1a to 4b.

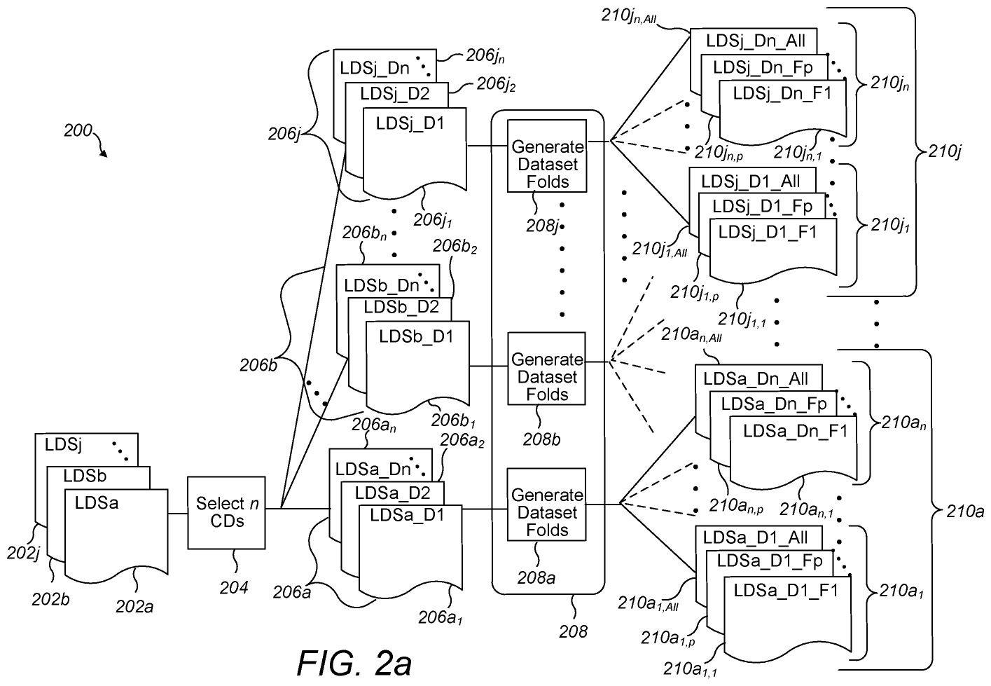

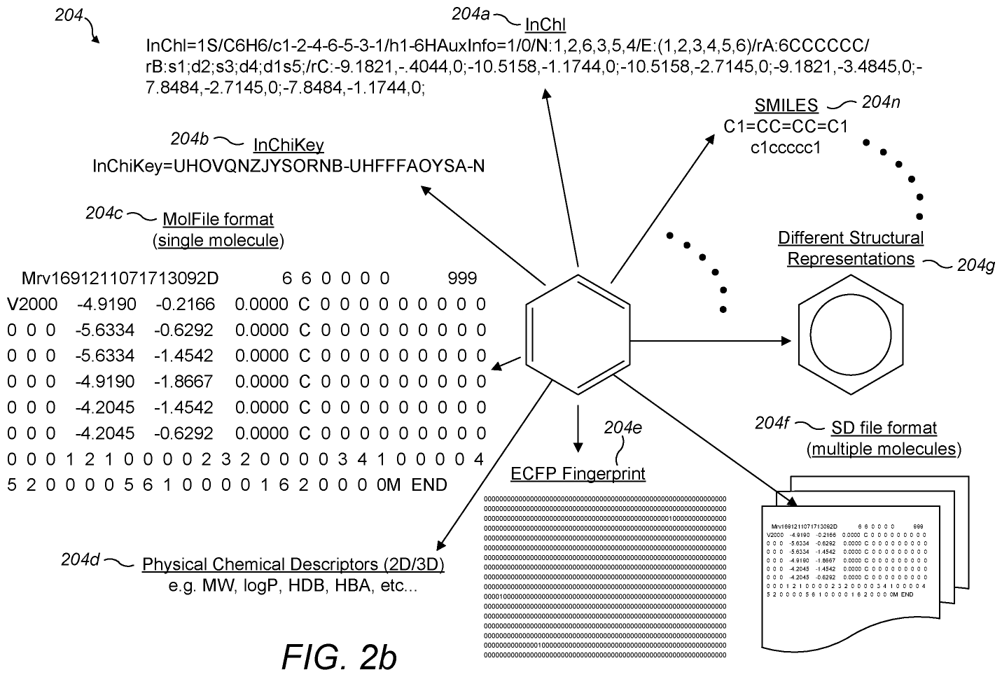

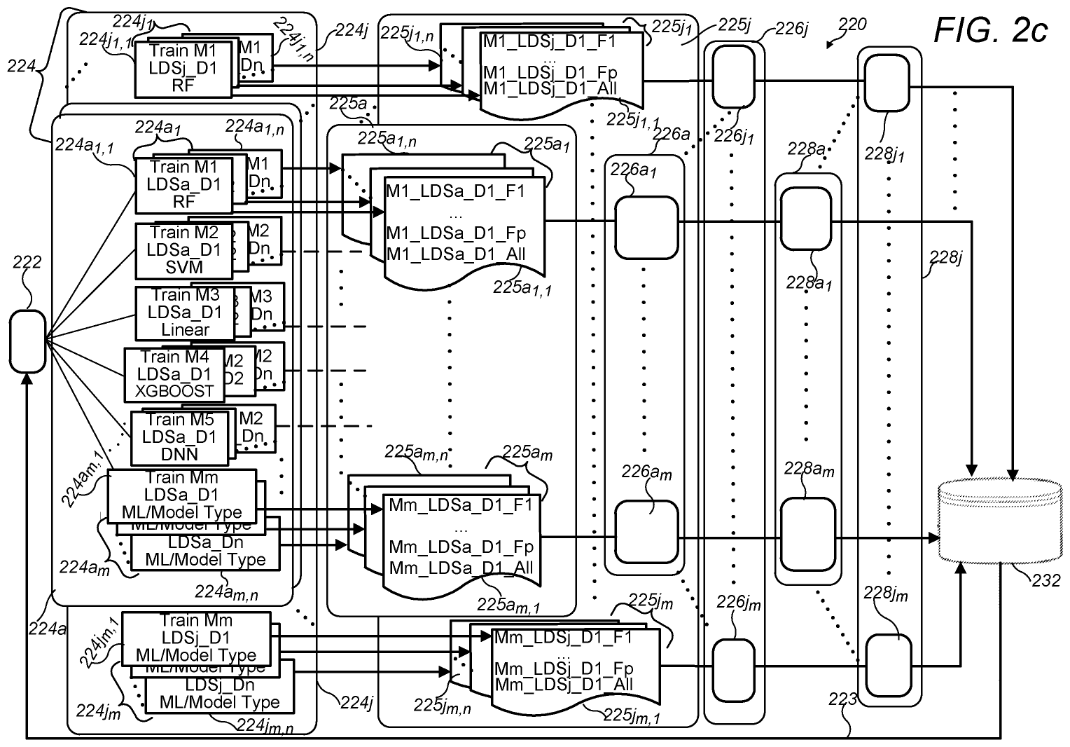

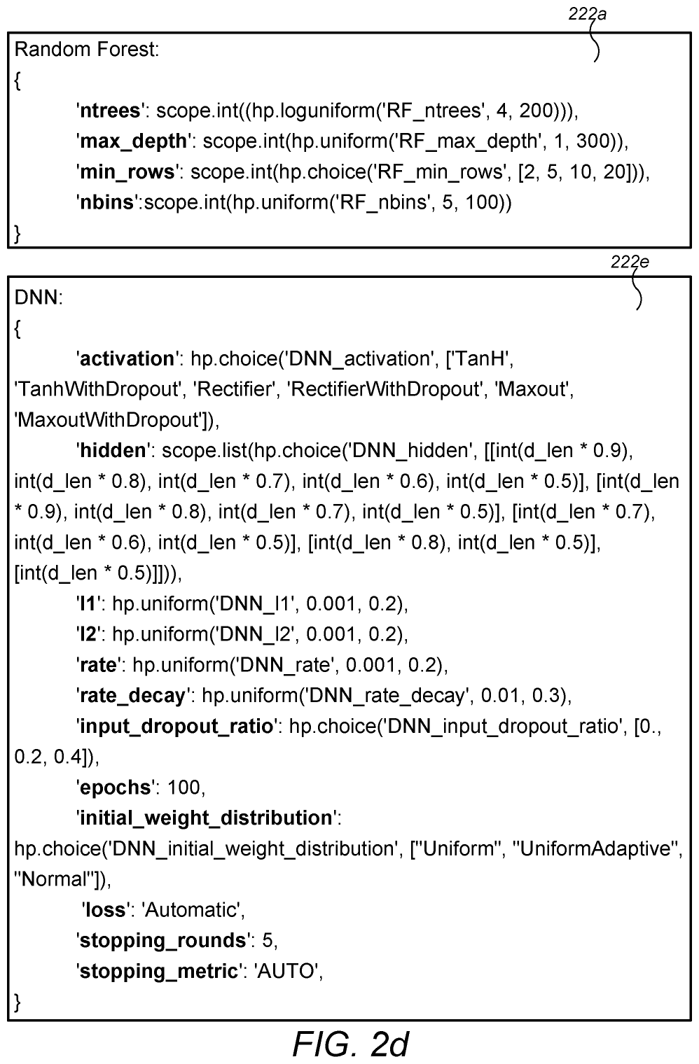

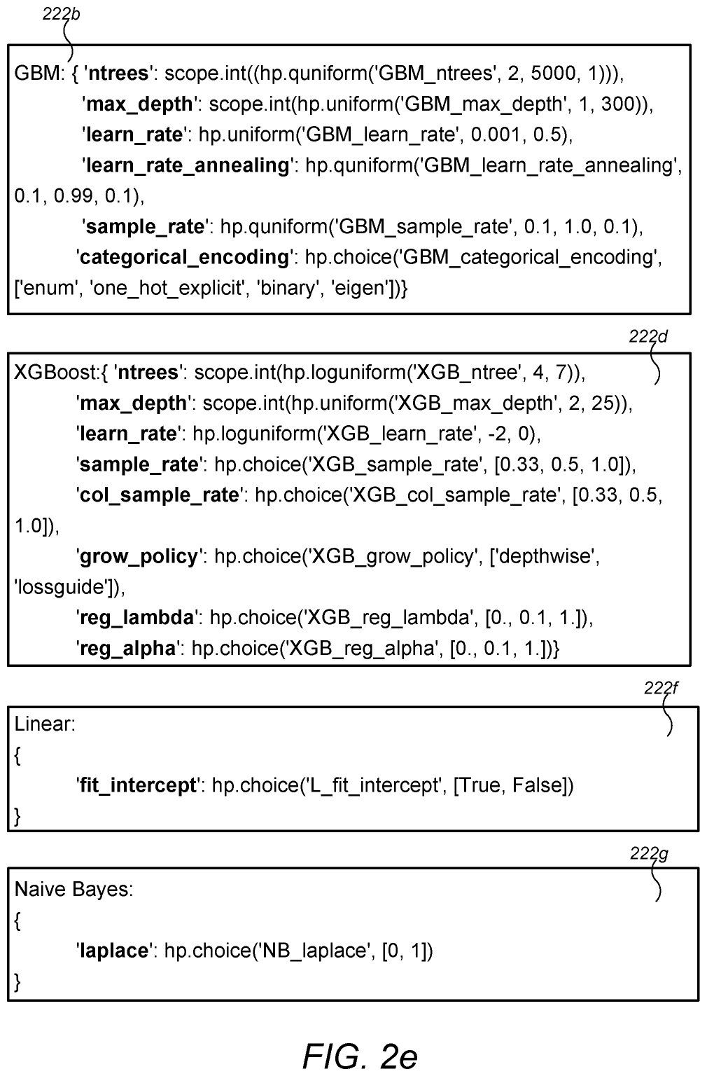

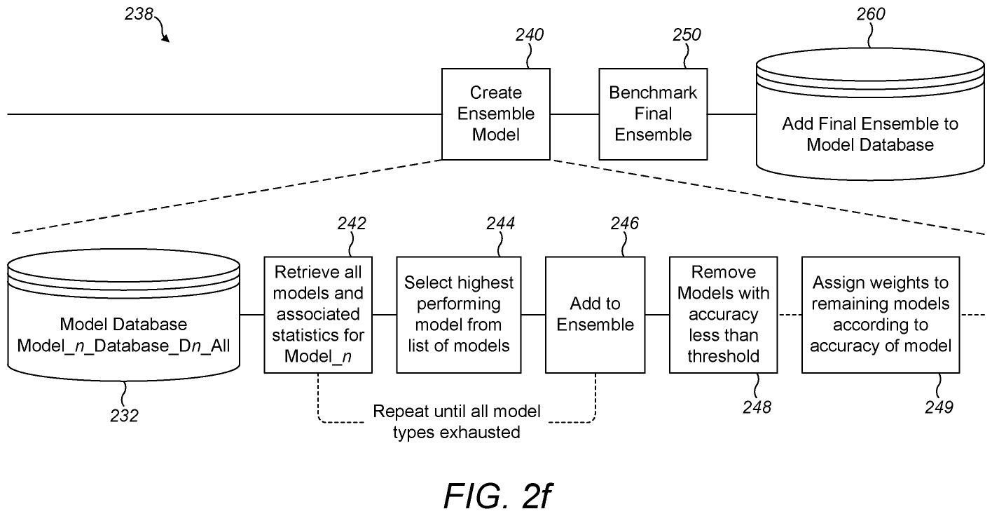

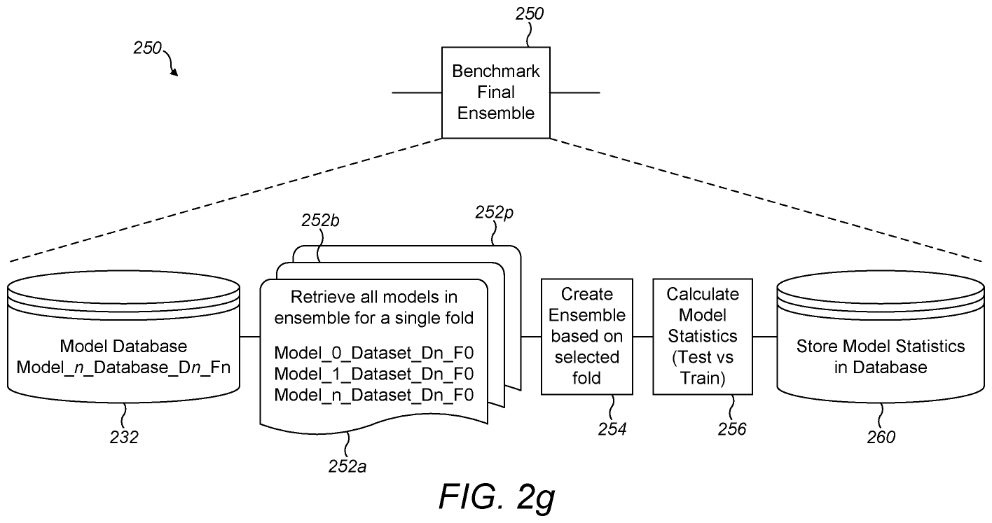

FIG. 2a is a schematic diagram illustrating an apparatus 200 for generating a plurality of datasets associated with compounds for use with the process 100 according to the invention. In this example, the plurality of datasets 210a-210j are generated from a set of j labelled datasets 202a-202j (e.g. LDSa, LDSb, . . . , LDSj) associated with compounds, that may be selected and/or retrieved in which each labelled dataset may be used in training models from a plurality of models. Each of the models configured towards a common objective and/or for modelling a particular process or solving a particular problem associated with compounds. Each of the plurality of models may be associated with modelling a process, problem and/or having a similar objective in the cheminformatics and/or bioinformatics fields.

The plurality of datasets 210a, . . . , 210j are generated from the labelled datasets 202a-202j based on selecting n chemical or compound descriptors (CDs) 204, where n>1, which are used to modify the labelled datasets 202a-202j to form a plurality of sets of CD labelled datasets 206a, 206b, . . . , 206j. Each of the plurality of sets of CD labelled datasets 206a, 206b, . . . , 206j are generated or partitioned 208a-208j into a plurality of dataset folds 210a₁, . . . , 210aₙ, 210j₁, . . . , 210jₙ, which form the plurality of datasets 210a, . . . , 210j for generating, training and/or assessing a plurality of models. The plurality of datasets 210a, . . . , 210j may be stored for later retrieval when generating, training, and/or assessing the plurality of models.

FIG. 2b describes a number of n chemical and/or compound descriptors 204 for, by way of example only but is not limited to, the organic chemical compound benzene. A chemical/compound descriptor or chemical/compound descriptor (CD) format (also known as molecular descriptors or topological descriptors) may comprise or represent any data or protocol representative of describing, representing and/or encoding compound or molecular information and/or the structure of one or more compound(s). Examples of CDs or CD formats may include, by way of example only but is not limited to, any one or more or a combination of the following: International Chemical Identifier, InChI 204a; InChIKey 204b; MoIFile format 204c; two dimensional Physical Chemical descriptors 204d; three dimensional Physical Chemical descriptors; XYZ file format; Extended Connectivity Fingerprint, ECFP 204e; Structure Data Format 204f; structural formula or representation of the compound 204g; Simplified Molecular Input Line Entry Specification, SMILES, strings or format 204n; SMILES arbitrary target specification or format; Chemical Mark-up Language format; and any other CD or CD format for describing, representing and/or encoding molecular information and/or structure(s) of compounds. Further examples of CDs or CD formats may include one or more CDs and/or CD formats associated with CD categories based on one or more of, or a combination of one or more of, by way of example only but not limited to, constitutional indices, ring descriptors, topological indices, walk and path counts, connectivity indices, information indices, 2D matrix-based descriptors, 2D autocorrelations, Burden-eigenvalues, P-VSA-like descriptors, ETA indices, edge adjacency indices, adjacency matrix descriptors, geometrical descriptors, 3D matrix-based descriptors, 3D autocorrelations, radial distribution function (RDF) descriptors, 3D-MoRSE descriptors, WHIM descriptors, GETAWAY descriptors, randic molecular profiles, atom-centred fragments, functional group counts, Atom-type E-state indices, CATS-2D, 2D atom pairs, 3D atom pairs, charge descriptors, molecular properties, drug-like indices, and any other CD/CD format or CD category for describing, representing and/or encoding molecular information and/or structure(s) of compounds.

Referring to FIGS. 2a and 2b, in order to optimise the input data format or descriptor used by the labelled datasets when training the models, each of the labelled datasets 202a-202j (e.g. LDSa, LDSb, LDSj) is used to generate a plurality of sets of CD labelled datasets 206a-206j using a number of n selected chemical or compound descriptors (CDs) 204a, 204b, . . . , 204n, where n>1, a plurality of CDs, (e.g. D1, D2, . . . , Dn). The n selected CDs are different to each other. Although the n selected CDs are different to each other, as there are many CDs per CD category two or more of the n selected CDs may belong to the same CD category.

For example, for labelled dataset 202a, a set of CD labelled datasets 206a based on the plurality of n CDs 204a, 204b, . . . , 204n can be generated. So, for labelled dataset 202a (e.g. LDSa) a set of CD labelled datasets 206a is generated based on the plurality of n CDs in which the set of CD labelled datasets 206a includes CD labelled datasets 206a₁, 206a₂, . . . , 206aₙ (e.g. LDSa_D1, LDSa_D2, . . . , LDSa_Dn); for labelled dataset 202b (e.g. LDSb) a set of CD labelled datasets 206b is generated based on the plurality of n CDs in which set of CD labelled datasets 206b includes CD labelled datasets 206b₁, 206b₂, . . . , 206bₙ (e.g. LDSb_D1, LDSb_D2, . . . , LDSb_Dn), and so on, and for labelled dataset 202j (e.g. LDSj) a set of CD labelled datasets 206j is generated based on the plurality of n CDs in which set of CD labelled datasets 206b includes CD labelled datasets 206j₁, 206j₂, 206jₙ (e.g. LDSj_D1, LDSj_D2, . . . , LDSj_Dn).

For example, for each of the plurality of CDs 204a, 204b, . . . , 204n, a copy of the labelled dataset 202a is generated and the data representative of the compounds associated with the copied labelled dataset 202a is formatted based on one of the CDs 204a, . . . , 204n to form a CD labelled dataset 206a₁ according to that CD 204a. Thus, a set of CD labelled datasets 206a is formed in which each dataset differs by the CD used to format the original labelled dataset 202a. For example, labelled dataset 202a may be copied n times, and each copied labelled dataset is “reformatted” by a different CD from the plurality of n CDs 204a-204n to form the set of CD labelled datasets 206a including CD labelled datasets 206a₁, 206a₂, 206aₙ; labelled dataset 202b may be copied n times, and each copied labelled dataset is “reformatted” by a different CD from the plurality of n CDs 204a-204n to form the set of CD labelled datasets 206b including CD labelled datasets 206b₁, 206b₂, . . . , 206bₙ; and so on including labelled dataset 202j, which may be copied n times, and each copied labelled dataset is “reformatted” by a different CD from the plurality of n CDs 204a-204n to form the set of CD labelled datasets 206j including CD labelled datasets 206j₁, 206j₂, . . . , 206jₙ.

In another example, each labelled dataset 202a may be used to generate a set of CD labelled datasets 206a based on a number of n CDs 204a-204n, n>1 or a plurality of CDs for generating the plurality of datasets 210a-210j. Each set of CD labelled datasets 206a includes the same labelled dataset 202a but being described by a different CD from the plurality of CDs 204a-204n. This may be achieved by replicating each labelled dataset 202a based on the number of the plurality of CDs 204a-204n, and then modifying the compounds described in each replicated labelled dataset 202a to be based on a different CD or compound input format selected from a plurality of CDs 204a-204n. As another example, the plurality of datasets may be generated from the set of labelled datasets 202a-202j in which groups of CD labelled datasets 206a-206j for each labelled dataset in the set of labelled datasets 202a-202j are generated based on a plurality of CDs 204a-204n, where each CD is different.

Once the plurality of sets of CD labelled datasets 206a, 206b, . . . , 206j are generated further datasets may be required for use in generating, training and/or assessing the plurality of models. For example, the plurality of models may be generated, trained and/or assessed based on, by way of example only but not limited to, p-fold cross-validation technique(s), where p>1. In this example, the models may be assessed using a p-fold cross-validation technique. P-fold cross-validation requires that each labelled dataset is partitioned or split into P different portions, where each portion is called a fold. Thus, a further P datasets are generated or formed from each labelled dataset. Cross-validating each of a plurality of models generally requires generating multiple folds for each labelled dataset in the sets of CS labelled datasets 206a-206j, training said each model on each of the multiple folds for that dataset to generate a MPS, and combining the MPSs from each fold to generate a combined MPS for that model and that dataset.

P-fold cross-validation may require that each labelled dataset is partitioned or split into P different portions, where each portion is called a fold. Each labelled dataset may be partitioned or split based on any splitting method such as, by way of example only but not limited to one or more from the group of: Random partitioning or splitting; splitting or partitioning by single property distribution; splitting or partitioning by multiple property distribution (MPO distribution); chemical scaffold based partitioning or splitting; partitioning/splitting based on time-splits; partitioning and/or splitting based on chemical similarity; splitting/partitioning using one or more clustering methods based on, by way of example only but not limited to, any of the above splitting methods; splitting/partitioning using chemical series based on, by way of example only but not limited to, any of the above splitting methods; any other splitting or partitioning method that ensures P folds of the labelled dataset are different from each other.

In particular, each of set of the plurality of CD labelled datasets 206a, 206b, . . . , 206j are passed through a dataset fold generator 208, which may include separate generators 208a-208j, that partition or split each of the datasets in each set of the plurality of CD labelled datasets 206a, 206b, . . . , 206j into a number of p different portions (e.g. p=5 folds of 80:20 splits), where p>1, to form the plurality of datasets 210a, . . . , 210j. For example, for CD labelled dataset 206a, each of the CD labelled datasets 206a₁, . . . , 206a, are passed through generator 208a, which generates a plurality of sets of dataset folds 210a₁, . . . , 210a, corresponding to the CD labelled datasets 206a₁, . . . , 206aₙ. Each of the sets of dataset folds 210a₁, . . . , 210aₙ include p CD labelled dataset folds and the entire CD labelled dataset. For example, the set of dataset folds 210a₁ includes p CD labelled dataset folds 210a_{1,1}, . . . , 210a_{1,p} and the entire CD labelled dataset 210a_{1,ALL}, which corresponds to the CD labelled dataset 206a₁.

For CD labelled dataset 206a, each of the CD labelled datasets 206a₁, . . . , 206aₙ are passed through generator 208a, which generates a plurality of sets of dataset folds 210a₁, . . . , 210aₙ corresponding to the CD labelled datasets 206a₁, . . . , 206aₙ. Each of the sets of dataset folds 210a₁, . . . , 210aₙ include p CD labelled dataset folds and the entire CD labelled dataset. CD labelled dataset 206a₁ (e.g. LDSa_D1) corresponding to CD 204a (e.g. D1) is partitioned into the set of dataset folds 210a₁ which includes p different CD labelled dataset folds 210a_{1,1}, . . . , 210a_{1,p} and the entire CD labelled dataset 210a_{1,ALL}, which corresponds to the CD labelled dataset 206a₁. Similarly, CD labelled dataset 206aₙ (e.g. LDSa_Dn) corresponding to CD 204n (e.g. Dn) is partitioned into the set of dataset folds 210a, which includes p different CD labelled dataset folds 210a_{n,1}, . . . , 210a_{n,p} and the entire CD labelled dataset 210a_{n,ALL}, which corresponds to the CD labelled dataset 206aₙ.

Similarly, for CD labelled dataset 206j, each of the CD labelled datasets 206j₁, . . . , 206jₙ are passed through generator 208j, which generates a plurality of sets of dataset folds 210j₁, . . . , 210jₙ corresponding to the CD labelled datasets 206j₁, . . . , 206jₙ. Each of the sets of dataset folds 210j₁, . . . , 210jₙ include p different CD labelled dataset folds and the entire CD labelled dataset. CD labelled dataset 206j, (e.g. LDSj_D1) corresponding to CD 204a (e.g. D1) is partitioned or portioned into the set of dataset folds 210j, which includes p different CD labelled dataset folds 210j_{1,1}, . . . , 210j_{1,p} and the entire CD labelled dataset 210j_{1,ALL}, which corresponds to the CD labelled dataset 206j₁. Similarly, CD labelled dataset 206j (e.g. LDSj_Dn) corresponding to CD 204n (e.g. Dn) is partitioned or portioned into a set of dataset folds 210j which includes p different CD labelled dataset folds 210j_{n,1}, . . . , 210j_{n,p} and the entire CD labelled dataset 210j_{n,ALL}, which corresponds to the CD labelled dataset 206jₙ.

As an example, for j=M labelled datasets, a number of n=N different CDs, and a number of p=P folds for cross-validation, then there will be a total of M·N·(P+1) datasets in the plurality of datasets 210a-210j. The plurality of datasets 210a-210l may be stored for later retrieval during generating, training and/or assessment of the plurality of models.

FIG. 2c is a schematic diagram illustrating an example model generating, training and assessment (MGTA) apparatus 220 for generating and training a plurality of set(s) of models 224a-224m and assessing a plurality of sets of trained models 225a-225m, which are selected to form a set of ‘optimal’ trained models for use with one or more ensemble models. The set of ‘optimal’ trained models are optimal in the sense that they satisfy one or more MPSs criteria or conditions. For example, the MPS(s) associated with a model meeting or being greater than one or more predetermined MPS(s) threshold(s). In another example, all models may be ranked according to their MPSs in which the best performing K models or topmost performing K models are selected for inclusion into the set of optimal trained models. The set of optimal trained models may be stored in a model database 232 for use in forming one or more ensemble models.

Referring to FIGS. 2a and 2c, the MGTA apparatus 220 includes a model generation/training (MGT) apparatus 224 that generates and trains a plurality of models 224a to 224j based on a number of factors including, by way of example only but is not limited to, the plurality of datasets 210a-210j associated with compounds and a number h of a plurality of sets of hyperparameters 222 associated with a number m of one or more ML technique(s), where m<=1 and m<=h and h is a multiple of m. These are used to generate and train the plurality of models 224a-224j. In this example, the MGTA system 220 searches over the plurality of datasets 210a-210j, a number h of a plurality of sets of hyperparameters 222, and one or more ML technique(s) to find the best performing trained models, which are stored as a set of optimal trained models for use in one or more ensemble models.

The MGTA apparatus 220 implements the search by performing a number of iterations over the plurality of sets of hyperparameters 222 in which each iteration selects a unique number of m sets of hyperparameters 222a-222m each corresponding to a number m of one or more ML technique(s) used to generate the models. The MGT apparatus 224 generates and trains one or more set(s) of models 224a-224j based on the selected m sets of hyperparameters 222a-22m and retrieving the plurality of datasets 210a-210j), which has been generated based on a number n of chemical or compound descriptors, and applying these to the m one or more ML technique(s) to output a plurality of sets of trained models 225a-225j. The calculation MPSs apparatus 226a, . . . , 226j calculates MPSs for the plurality of sets of trained models 225a-225j. These MPSs are sent to model assessment devices 228a-228j for determining, for the current iteration, which models of the plurality of sets of trained models 225a-225j may be selected and stored in model database 232 as a set of optimal trained models. The model assessment device 228a-228j use one or more criteria or conditions based on the MPSs to make a determination as to whether a model from the plurality of sets of trained models 225a-225j will be selected to be part of the set of optimal trained models, which may be stored in model database 232. Once all of the plurality of sets of trained models 225a-225j have been assessed, the MGTA apparatus 220 performs another iteration by selecting another unique number of m sets of hyperparameters 222a-222m, different from the previous iterations, in which each correspond to a number m of the one or more ML technique(s) used to generate the models. The number of iterations that are performed may be predetermined, or simply based on the number of unique sets of m sets of hyperparameters 222a-222m in the plurality of sets of hyperparameters 222.

FIGS. 2d and 2e are tables describing example hyperparameters for several example ML techniques that may be used to generate one or more model(s). Prior to an ML technique defining and generating a model via training, the ML technique is initialised based on one or more hyperparameters or a set of hyperparameters associated with the ML technique and problem or process to be modelled. A set of hyperparameters corresponding to a ML technique contains various predefined parameters, the values of which define and/or affect the operation of the ML technique during training and generation of the model based on the ML technique. The parameter values of each hyperparameter in the set of hyperparameters for that ML technique will affect the operation of the ML technique during training and generation of the model. Even minor changes to the parameter values of each hyperparameter can affect the operation of the same ML technique differently during training and generation of the model. This results in a different model for each different set of hyperparameters that has one or more changed hyperparameter values compared with another set of hyperparameters for the same ML technique.

FIGS. 2d and 2e describe an example selection of hyperparameter sets for various example ML techniques such as, by way of example only but not limited to, random forest (RFs) hyperparameter set 222a, deep neural network (DNN) hyperparameter set 222e, gradient boosting machine (GBM) hyperparameter set 222b, XGBoost hyperparameter set 222d, Linear hyperparameter set 222f, and Nave Bayes hyperparameter set 222g. It is noted that each example shows only a selection of the possible hyperparameters for each set that may be used by that ML technique.

For example, the RF ML technique may use a set of RF hyperparameters 222a that includes, by way of example only but is not limited to: 1) ‘ntrees’ hyperparameter defines the number of RF trees, which may, in this example, have a parameter value in the range from, by way of example only but is not limited to, 4 to 200; 2) ‘max_depth’ hyperparameter defines the maximum node depth of each RF tree, and may have a parameter value in the range from, by way of example only but is not limited to, 1 to 300; 3) ‘min_rows’ hyperparameter defines the fewest allowed (weighted) observations in a leaf of the RF tree, which may, in this example, have a parameter value in the range, by way of example only but is not limited to, [2, 5, 10, 20]; and 4) ‘nbins’ hyperparameter defines the RF tree builds a histogram with this number of bins, which may, in this example, be in the range from, by way of example only but is not limited to, 5 to 100.

For example, the deep neural network (DNN) ML technique may use a set of DNN hyperparameters 222e that includes, by way of example only but is not limited to: 1) ‘activation’ hyperparameter defines the activation function between input and output of each node in the DNN, which may, in this example, have a parameter value based on an activation function such as, by way of example only but not limited to, ‘Tan H’, ‘Tan hWithDropout’, ‘Rectifier’, ‘RectifierWithDropout’, ‘Maxout’, ‘MaxoutWithDropout’; 2) ‘hidden’ hyperparameter defines the number of hidden layers or hidden units per hidden layer for the DNN, which may be any integer greater than or equal to 1, e.g. in the range of, by way of example only but is not limited to, 1 to 4; 3) ‘I1’ hyperparameter defining whether I1 regularisation is used and the Lagrange multipliers, which may, in this example, be in the range of, by way of example only but is not limited to, 0.001 to 0.2; 4) ‘I2’ hyperparameter defines whether I2 regularisation is used and the Lagrange multipliers, which may, in this example, be in the range of, by way of example only but is not limited to, 0.001 to 0.2; 5) ‘rate’ hyperparameter defines the learning rate of the DNN, which may, in this example be in the range of, by way of example only but is not limited to, 0.001 to 0.2; 6) ‘rate_decay’ hyperparameter defines the rate at which the learning rate decays, which may, in this example, be in the range of, by way of example only but is not limited to, 0.01 to 0.3; 7) ‘input_dropout_ratio’ hyperparameter defines the proportion of nodes that are set to zero to prevent overfitting, which may, in this example, have parameter values in the range of, by way of example only but is not limited to, 0 to 0.4; 8) ‘epochs’ hyperparameter defines the number of passes through a given dataset, which may, in this example, have any parameter value such as, by way of example only but not limited to, 100; 9) ‘initial_weight_distribution’ hyperparameter defines the distribution that the initial weights of the DNN may be set to, which may, in this example, include one or more distributions such as, by way of example only but is not limited to, “Uniform”, “UniformAdaptive”, “Normal” distributions; 10) ‘loss’ hyperparameter may define the loss function, which may, in this example be set to being ‘Automatic’ chosen, or ‘manually’ chosen; 11) ‘stopping_rounds’ hyperparameter defines the number of training iterations, which may, in this example be any suitable integer value, by way of example only but is not limited to, 5; 12) ‘stopping_metric’ hyperparameter defines the type of stopping metric for ending the training of the DNN, which may, in this example, be selected to be ‘AUTO’.

For example, the GBM ML technique may use a set of GBM hyperparameters 222b that includes, by way of example only but is not limited to: 1) the ‘ntrees’ hyperparameter defining the number of GBM trees, which may, in this example, have parameter values in the range of, by way of example only but is not limited to, 2 to 5000; 2) the ‘max_depth’ hyperparameter defining the maximum node depth of each GBM tree, which may, in this example, have parameter values in the range of, by way of example only but is not limited to, 1 to 300; 3) the ‘learn_rate’ hyperparameter defines the learning rate of the GBM, which may, in this example be in the range of, by way of example only but is not limited to, 0.001 to 0.5; 4) the ‘learn_rate_annealing’ hyperparameter defining, which may, in this example be in the range of, by way of example only but is not limited to, 0.1 to 0.99; 5) the ‘sample_rate’: hyperparameter defining the GBM sampling rate, which may, in this example be in the range of 0.1 to 1.0; 6) the ‘categorical_encoding’ hyperparameter that may define the categorical encoding of the output of the GBM, which may, in this example, be selected from a list of categorical encoding types such as, by way of example only but is not limited to, ‘enum’, ‘one_hot_explicit’, ‘binary’, and ‘eigen’.

For example, the XGBoost ML technique may use a set of XGBoost hyperparameters 222d that includes, by way of example only but is not limited to: 1) the ‘ntrees’ hyperparameter defining the number of XGB trees, which may, in this example, have parameter values in the range of, by way of example only but is not limited to, 4 to 7; 2) the ‘max_depth’ hyperparameter defining the maximum node depth of each XGB tree, which may, in this example, have parameter values in the range of, by way of example only but is not limited to, 2 to 25; 3) the ‘learn_rate’ hyperparameter defines the learning rate of XGBoost, which may, in this example, be a parameter value in the range of, by way of example only but is not limited to, −2 to 0; 4) the ‘sample_rate’: hyperparameter defining the XGB sampling rate, which may, in this example be in the range of 0 to 1.0; 5) the ‘col_sample_rate’ hyperparameter defining the column sampling rate, which may, in this example, be a parameter value in the range of, by way of example only but is not limited to, 0 to 1.0; 6) the ‘grow_policy’ hyperparameter defining the tree growing policy controlling the way new nodes are added to the tree, which may, in this example, be a parameter value in selected from the list of, by way of example only but is not limited to, ‘depthwise’, ‘Iossguide’; 7) the ‘reg_lambda’ hyperparameter defining the lambda regularisation parameter, which may, in this example, be a parameter value in the range of, by way of example only but is not limited to, 0 to 1; and 8) the ‘reg_alpha’ hyperparameter defining the alpha regularisation parameter, which may, in this example, be a parameter value in the range of, by way of example only but is not limited to, 0 to 1.

For example, the Linear ML technique may use a set of Linear hyperparameters 222f that includes, by way of example only but is not limited to, a ‘fit_intercept’ hyperparameter, which may, in this example, have a parameter value that is selected as either True or False. The Nave Bayes ML technique may use a set of Nave Bayes hyperparameters 222g that includes, by way of example only but is not limited to, the laplace hyperparameter, which may, in this example, be have a parameter value in the range of, by way of example only but not limited to, 0 to 1.

As can be seen, each ML technique uses a different set of hyperparameters, in which each of the hyperparameters can have a different possible number of values. Since each hyperparameter in a set of hyperparameters may have a range of parameter values, this means that there are a large number of different unique sets of hyperparameters for the same ML technique that can generate a similarly large number of different models. For example, for a ML technique that has a number of H hyperparameters, in which the i-th hyperparameter has number of hᵢ possible parameter values for 1<=i<=H, then there is a number of Πᵢ₌₁^{H}, hᵢ possible sets of hyperparameters for that particular ML technique. Furthermore, if there is a number of M different ML technique(s) in which the m-th ML technique has a number of Hₘ hyperparameters, where each of the Hₘ hyperparameters has a number of h_{i,m} possible parameter values for 1<=i<=Hₘ and 1<=m<=M, then there will be a number of, or a plurality of, Σₘ₌₁^{H}øᵢ₌₁^{H}^{ₘ}h_{i,m} possible sets of hyperparameters. Thus, the chances of finding the set of hyperparameters for a number M of ML technique(s) that generates the best or optimal model(s) for modelling a problem and/or process over a number of training datasets decreases as the number of hyperparameters, the number of possible parameters values for each hyperparameter, and the number of ML techniques under consideration increases.

Referring to FIG. 2c, the MGTA 220 may generate a plurality of sets of hyperparameters and corresponding possible parameter values or a number of m ML techniques, where m>=1 (e.g. one or more ML technique(s) or a plurality of ML techniques) that may be used to find one or more of the best performing models. The MGTA 220 may then perform a search of optimal models by iterating the training and generation of models based on one or more ML techniques over the plurality of sets of hyperparameters for each ML technique. Hyperparameter selection 222 may be performed in each iteration, where a unique number of m sets of hyperparameters 222a-222m are selected, each of the sets of hyperparameters 222a-222m corresponding to each of the number m of one or more ML technique(s) used to train and generate one or more models for assessment.

In each iteration, the hyperparameter selection 222 is performed over a plurality of hyperparameters, where a set of hyperparameters 222a-222m is selected for each ML technique. Each selected set of hyperparameters 222a-222m being a unique combination from the possible parameter values for each of the hyperparameters of that set. Thus, a number of m sets of hyperparameters 222a-222m may be selected from the plurality of hyperparameters 222 for input to the corresponding one or more of the m ML technique(s) for training the corresponding ML techniques and generating the one or more set(s) of trained models 225a-225j.

The MGT apparatus 224 takes as input, the plurality of datasets 210a-210j and the selected number of m sets of hyperparameters 222a-222m, each set of hyperparameters corresponding to one of the m ML technique(s) which are input to model generator/training apparatus 224. In this example, the number of m ML techniques includes, by way of example only but is not limited to, a RF ML technique, an SVM ML technique, a Linear ML technique, an XGBoost ML technique, a DNN ML technique, and any other type of ML technique that may be used to generate a plurality of models for assessment.

As described in FIG. 2b, the plurality of datasets 210a-210j includes a plurality of sets of CD labelled datasets 206a, . . . , 206j. Each of the sets of CD labelled datasets 206a, . . . , 206j includes a plurality of CD labelled datasets 206a₁, . . . , 206aₙ, . . . , 206j₁, . . . , 206jₙ. For example, the set of CD labelled datasets 206a includes the plurality of CD labelled datasets 206a₁, . . . , 206aₙ, and the set of CD labelled datasets 206j includes the plurality of CD labelled datasets 206ⱼ₁, . . . , 206jₙ. Each of the plurality of CD labelled datasets 206a₁, . . . , 206aₙ for each set of CD labelled dataset 206a have been partitioned into a plurality of sets of CD labelled dataset folds 210a₁, . . . , 210aₙ, in which each of the sets of CD labelled dataset folds 210a₁, . . . , 210aₙ comprises a plurality of CD labelled dataset folds 210a_{1,1}, . . . , 210a_{1,p}, . . . , 210a_{n,1}, . . . , 210a_{n,p}. For example, each of the plurality of CD labelled datasets 206a₁, . . . , 206aₙ for the set of CD labelled datasets 206a has been partitioned into sets of CD labelled dataset folds 210a₁, . . . , 210aₙ, where each set of CD labelled dataset folds 210a₁ includes a plurality of CD labelled dataset folds 210a_{1,1}, . . . , 210a_{1,p} and 210a_{1,All}. Each of the plurality of CD labelled datasets 206j₁, . . . , 206jₙ for the set of CD labelled datasets 206j has been partitioned into sets of CD labelled dataset folds 210j₁, . . . , 210jₙ, where each set of CD labelled dataset folds 210j₁ includes a plurality of CD labelled dataset folds 210j_{1,1}, . . . , 210j_{1,p} and 210j_{1,All}. Thus, the plurality of datasets 210a-210j includes a plurality of CD labelled dataset folds 210a_{1,1}, . . . , 210a_{1,p}, . . . , 210j_{1,1}, . . . , 210j_{1,p}.

Referring to FIG. 2c, the MGT 224 includes MGTs 224a-224j for each of the sets of CD labelled datasets 206a, . . . , 206j, each of which is used for train one of m types of ML technique(s), m>=1, to generate a corresponding plurality of sets of trained models 225a-225j. For example, MGT 224a receives the set of CD labelled datasets 206a along with the selected sets of hyperparameters 222a-222m for each of the m types of ML techniques that have been selected for use in training and generating one or more trained models. Similarly, MGT 224j receives the set of CD labelled datasets 206j along with the selected sets of hyperparameters 222a-222m for each of the m types of ML techniques that have been selected for use in training and generating one or more trained models. This may be performed on the ML techniques based on, by way of example only but not limited to, SVM, Linear, XGBoost, DNN and any other type of ML technique. It is to be appreciated by the skilled person that any other one or more ML technique(s) or combinations thereof may be used.

For example, MGT 224a retrieves the set of CD labelled datasets 206a to generate a plurality of trained models 225a by training each of m sets of ML techniques 224a₁, . . . , 224aₘ on each corresponding CD labelled dataset of the set of CD labelled datasets 206a, which comprises the plurality of CD labelled datasets 206a₁, . . . , 206aₙ that correspond to the set of CD labelled dataset folds 210a₁, . . . , 210aₙ. Each set of CD labelled dataset folds 210a₁, . . . , 210aₙ comprises a plurality of CD labelled dataset folds. For example, the set of CD labelled dataset folds 210a₁ includes the plurality of CD labelled dataset folds 210a_{1,1}, . . . , 210a_{1,p} and 210a_{1,All}; the set of CD labelled dataset folds 210aₙ includes the plurality of CD labelled dataset folds 210a_{n,1}, . . . , 210a_{n,p} and 210a_{n,All}. Each of the sets of ML techniques 224a₁, . . . , 224aₘ is based on the same type of ML technique configured with the corresponding selected set of hyperparameters 222a-222m but trained on a different one of the n datasets from the set of CD labelled datasets 206a to generate corresponding sets of trained models 225a₁, . . . , 225aₘ.

Similarly, MGT 224j retrieves the set of CD labelled datasets 206j and generates a plurality of trained models 225j by training each of m sets of ML techniques 224j₁, . . . , 224jₘ on each corresponding CD labelled dataset of the set of CD labelled datasets 206j, which comprises the plurality of CD labelled datasets 206j₁, . . . , 206jₙ that correspond to the set of CD labelled dataset folds 210j₁, . . . , 210jₙ. Each set of CD labelled dataset folds 210j₁, . . . , 210jₙ comprises a plurality of CD labelled dataset folds. For example, the set of CD labelled dataset folds 210j₁ includes the plurality of CD labelled dataset folds 210j_{1,1}, . . . , 210j_{1,p} and 210j_{1,All}; the set of CD labelled dataset folds 210jₙ includes the plurality of CD labelled dataset folds 210j_{n,1}, . . . , 210j_{n,p} and 210j_{n,All}. Each of the sets of ML techniques 224j₁, . . . , 224jₘ comprises the same type of ML technique configured with the corresponding selected set of hyperparameters 222a-222m but trained on one of the n datasets from the set of CD labelled datasets 206j to generate corresponding sets of trained models 225j₁, . . . , 225jₘ.

For example, the set of ML techniques 224a₁ are based, in this example, on the RF ML technique and includes a number of n groups of ML techniques 224a_{1,1}, . . . , 224a_{1,n}, in which each of the groups of ML techniques 224a_{1,1}, . . . , 224a_{1,n}, is based on the RF ML technique and has been configured with the selected set of RF hyperparameters 222a and is to be trained on the set of CD labelled datasets 206a, which includes the plurality of CD labelled datasets 206a₁, . . . , 206aₙ (e.g. LDSa_D1, . . . , LDSa_Dn). Similarly, the set of ML techniques 224j₁ are based, in this example, on the RF ML technique and includes a number of n groups of ML techniques 224j_{1,1}, . . . , 224j_{1,n}, in which each of the groups of ML techniques 224j_{1,1}, . . . , 224j_{1,n} is also based on the RF ML technique and has been configured with the selected set of RF hyperparameters 222a and is to be trained on the set of CD labelled datasets 206j, which includes the plurality of CD labelled datasets 205j₁, . . . , 206jₙ (e.g. LDSj_D1, . . . , LDSj_Dn).

In MGT 224a, each group of the n groups of ML techniques 224a_{1,1}, . . . , 224a_{1,n} is trained on a corresponding different CD labelled dataset from the plurality of CD labelled datasets 206a₁, . . . , 206aₙ (e.g. LDSa_D1, . . . , LDSa_Dn). Each of the groups of ML techniques 224a_{1,1}, . . . , 224a_{1,n} in the set of ML technique(s) 224a₁ is trained based on the corresponding datasets of the set of CD labelled datasets 206a, which comprises the plurality of CD labelled datasets 206a₁, . . . , 206aₙ, to generate a corresponding set of trained model(s) 225a₁. The set of trained model(s) 225a₁ comprises a number of n groups of trained model(s) 225a_{1,1}, . . . , 225a_{1,n}, each group corresponding to one of the trained groups of ML technique(s) 224a_{1,1}, . . . , 224a_{1,n}.

In MGT 224j, each group of the n groups of ML techniques 224j_{1,1}, . . . , 224j_{1,n} is trained on a corresponding different CD labelled dataset from the plurality of CD labelled datasets 206j₁, . . . , 206jₙ (e.g. LDSj_D1, . . . , LDSj_Dn). Each of the groups of ML techniques 224j_{1,1}, . . . , 224j_{1,n} in the set of ML technique(s) 224j₁ is trained based on the corresponding datasets of the set of CD labelled datasets 206j, which comprises the plurality of CD labelled datasets 206j₁, . . . , 206jₙ, to generate a corresponding set of trained model(s) 225j₁. The set of trained model(s) 225j₁ comprises a number of n groups of trained model(s) 225j_{1,1}, . . . , 225j_{1,n}, each group corresponding to one of the trained groups of ML technique(s) 224j_{1,1}, . . . , 224j_{1,n}.

Similarly, for MGT 224a, the set of ML techniques 224aₘ includes a number of n groups of ML techniques 224a_{m,1}, . . . , 224a_{m,n} of a particular selected ML type, in which each of the groups of ML techniques 224a_{m,1}, . . . , 224a_{m,n} has been configured with the selected set of hyperparameters 222m for that ML type and is to be trained on the set of CD labelled datasets 206a, which includes the plurality of CD labelled datasets 206a₁, . . . , 206aₙ (e.g. LDSa_D1, . . . , LDSa_Dn). Each group of the n groups of ML techniques 224a_{m,1}, . . . , 224a_{m,n} is trained on a corresponding CD labelled dataset from the plurality of CD labelled datasets 206a₁, . . . , 206aₙ (e.g. LDSa_D1, . . . , LDSa_Dn). Each of the groups of ML techniques 224a_{m,1}, . . . , 224a_{m,n} in the set of ML technique(s) 224aₘ is trained based on the corresponding datasets of the set of CD labelled datasets 206a, which comprises the plurality of CD labelled datasets 206a₁, . . . , 206aₙ, to generate a corresponding set of trained model(s) 225aₘ. The set of trained model(s) 225aₘ comprises a number of n groups of trained model(s) 225a_{m,1}, . . . , 225a_{m,n}, each group corresponding to one of the trained groups of ML technique(s) 224a_{m,1}, . . . , 224a_{m,n}.

Similarly, for MGT 224j, the set of ML techniques 224jₘ includes a number of n groups of ML techniques 224j_{m,1}, . . . , 224j_{m,n} of a particular selected ML type, in which each of the groups of ML techniques 224j_{m,1}, . . . , 224j_{m,n} has been configured with the selected set of hyperparameters 222m for that ML type and is to be trained on the set of CD labelled datasets 206j, which includes the plurality of CD labelled datasets 206j₁, . . . , 206jₙ (e.g. LDSj_D1, . . . , LDSj_Dn). Each group of the n groups of ML techniques 224j_{m,1}, . . . , 224j_{m,n} is trained on a corresponding CD labelled dataset from the plurality of CD labelled datasets 206j₁, . . . , 206jₙ (e.g. LDSj_D1, . . . , LDSj_Dn). Each of the groups of ML techniques 224j_{m,1}, . . . , 224j_{m,n} in the set of ML technique(s) 224jₘ is trained based on the corresponding datasets of the set of CD labelled datasets 206j, which comprises the plurality of CD labelled datasets 206j₁, . . . , 206jₙ, to generate a corresponding set of trained model(s) 225jₘ. The set of trained model(s) 225jₘ comprises a number of n groups of trained model(s) 225j_{m,1}, . . . , 225j_{m,n}, each group corresponding to one of the trained groups of ML technique(s) 224j_{m,1}, . . . , 224j_{m,n}.

Referring to MGT 224a, for the set of ML techniques 224a₁, each of the groups of ML techniques 224a_{1,1}, . . . , 224a_{1,n}, further includes one or more ML technique(s) each of which are configured according to the same set of hyperparameters 222a but which are trained on different folds of the corresponding CD labelled datasets 206a₁, . . . , 206aₙ (e.g. LDSa_D1, . . . , LDSa_Dn). Each of the plurality of CD labelled datasets 206a₁, . . . , 206aₙ corresponds to a different set of CD labelled dataset folds 210a₁, . . . , 210aₙ, each set of folds 210a₁ corresponds to a plurality of CD labelled dataset folds 210a_{1,1}, . . . , 210a_{1,p} and 210a_{1,All}. Given each set of CD labelled dataset folds 210a₁ may have (p+1) folds, then each group of the groups of ML technique(s) 224a_{1,1}, . . . , 224a_{1,n} in the set of ML technique(s) 224a₁ includes (p+1) ML technique(s), each of which is trained on different ones of a plurality of CD labelled dataset folds 210a_{1,1}, . . . , 210a_{1,p} and 210a_{1,All} corresponding to said each set of CD labelled dataset folds 210a₁. This is performed for each of the sets of CD labelled dataset folds 210a₁, . . . , 210aₙ, which results in a set of trained models 225a₁ comprising the n groups of trained models 225a_{1,1}, . . . , 225a_{1,n}, in which each group of trained models 225a_{1,1}, . . . , 225a_{1,n} includes multiple trained models (e.g. a first group of trained models based on ML technique M1 may be represented by M1_LDSa_D1_F1, M1_LDSa_D1_F2, . . . , M1_LDSa_D1_Fp, and M1_LDSa_D1_All) based on the corresponding sets of CD labelled dataset folds 210a₁, . . . , 210aₙ.

Similarly, for the set of ML techniques 224aₘ, each of the groups of ML techniques 224a_{m,1}, . . . , 224a_{m,n} further includes one or more ML technique(s) each of which are configured according to the same set of hyperparameters 222m but which are trained on different folds of the corresponding CD labelled datasets 206a₁, . . . , 206aₙ (e.g. LDSa_D1, . . . , LDSa_Dn). Each of the plurality of CD labelled datasets 206a₁, . . . , 206aₙ corresponds to a different set of CD labelled dataset folds 210a₁, . . . , 210aₙ, each set of folds 210a₁ corresponds to a plurality of CD labelled dataset folds 210a_{1,1}, . . . , 210a_{1,p} and 210a_{1,All}. Given each set of CD labelled dataset folds 210a₁ may have (p+1) folds, then each group of the groups of ML technique(s) 224a_{m,1}, . . . , 224a_{m,n} in the set of ML technique(s) 224aₘ includes (p+1) ML technique(s), each of which is trained on different ones of a plurality of CD labelled dataset folds 210a_{1,1}, . . . , 210a_{1,p} and 210a_{1,All} corresponding to said each set of CD labelled dataset folds 210a₁. This is performed for each of the sets of CD labelled dataset folds 210a₁, . . . , 210aₙ, which results in a set of trained models 225aₘ comprising the n groups of trained models 225a_{m,1}, . . . , 225a_{m,n}, in which each group of trained models 225a_{m,1}, . . . , 225a_{m,n} includes multiple trained models (e.g. a first group of trained models based on ML technique Mm may be represented by Mm_LDSa_D1_F1, Mm_LDSa_D1_F2, . . . , Mm_LDSa_D1_Fp, and Mm_LDSa_D1_All) based on the corresponding sets of CD labelled dataset folds 210a₁, . . . , 210aₙ.

As an example, in the group of ML techniques 224a_{1,1} (e.g. RF ML technique) is trained on the set of CD labelled datasets 206a₁ (e.g. LDSa_D1), which comprises the set of CD labelled dataset folds 210a_{1,1}, . . . , 210a_{1,p} and 210a_{1,All}. This means that the group of ML techniques 224a_{1,1} includes (p+1) trained ML techniques based on RF ML technique in which each ML technique is configured with same hyperparameters 222a but trained on a different CD labelled dataset fold of the plurality of CD labelled dataset folds 210a_{1,1}, . . . , 210a_{1,p} and 210a_{1,All}. Training the group of ML techniques 224a_{1,1} on the plurality of CD labelled dataset folds 210a_{1,1}, . . . , 210a_{1,p} and 210a_{1,All}, thus generates a corresponding group of trained models 225a_{1,1} (e.g. M1_LDSa_D1_F1, M1_LDSa_D1_F2, . . . , M1_LDSa_D1_Fp, and M1_LDSa_D1_All), which includes (p+1) trained models for the CD labelled dataset folds 210a_{1,1}, . . . , 210a_{1,p} and 210a_{1,All}. Similarly, the group of ML techniques 224a_{1,n} (e.g. RF ML technique) is trained on the set of CD labelled datasets 206aₙ (e.g. LDSa_Dn), which comprises the set of CD labelled dataset folds 210a_{n,1}, . . . , 210a_{n,p} and 210a_{n,All}. This means that the groups of ML technique 224a_{1,n} are each trained on a corresponding CD labelled dataset fold of the CD labelled dataset folds 210a_{n,1}, . . . , 210a_{n,p} and 210a_{n,All}. This generates the group of trained models 225a_{1,n}, which includes (p+1) trained models each corresponding one of the CD labelled dataset folds 210a_{n,1}, . . . , 210a_{n,p} and 210a_{n,All}.

Referring to MGT 224j, each of the groups of ML techniques 224j_{1,1}, . . . , 224j_{1,n} in the set of ML technique(s) 224j₁ further includes one or more ML technique(s) each of which are configured according to the same set of hyperparameters 222a but which are trained on different folds of the corresponding CD labelled datasets 206j₁, . . . , 206jₙ (e.g. LDSj_D1, . . . , LDSj_Dn). Each of the plurality of CD labelled datasets 206j₁, . . . , 206jₙ corresponds to a different set of CD labelled dataset folds 210j₁, . . . , 210jₙ, each set of folds 210j₁ corresponds to a plurality of CD labelled dataset folds 210j_{1,1}, . . . , 210j_{1,p} and 210j_{1,All}. Given each set of CD labelled dataset folds 210j₁ may have (p+1) folds, then each group of the groups of ML technique(s) 224j_{1,1}, . . . , 224j_{1,n} in the set of ML technique(s) 224j₁ includes (p+1) ML technique(s), each of which is trained on different ones of a plurality of CD labelled dataset folds 210j_{1,1}, . . . , 210j_{1,p} and 210j_{1,All} corresponding to said each set of CD labelled dataset folds 210j₁. This is performed for each of the sets of CD labelled dataset folds 210j₁, . . . , 210jₙ, which results in a set of trained models 225j₁ comprising the n groups of trained models 225j_{1,1}, . . . , 225j_{1,n}, in which each group of trained models 225j_{1,1}, . . . , 225j_{1,n} includes multiple trained models (e.g. a first group of trained models based on ML technique M1 may be represented by M1_LDSj_D1_F1, M1_LDSj_D1_F2, . . . , M1_LDSj_D1_Fp, and M1_LDSj_D1_All) based on the corresponding sets of CD labelled dataset folds 210j₁, . . . , 210jₙ.

Similarly, for the set of ML techniques 224jₘ, each of the groups of ML techniques 224j_{m,1}, . . . , 224j_{m,n} further includes one or more ML technique(s) each of which are configured according to the same set of hyperparameters 222m but which are trained on different folds of the corresponding CD labelled datasets 206j₁, . . . , 206jₙ (e.g. LDSj_D1, . . . , LDSj_Dn). Each of the plurality of CD labelled datasets 206j₁, . . . , 206jₙ corresponds to a different set of CD labelled dataset folds 210j₁, . . . , 210jₙ, each set of folds 210j₁ corresponds to a plurality of CD labelled dataset folds 210j_{1,1}, . . . , 210j_{1,p} and 210j_{1,All}. Given each set of CD labelled dataset folds 210j₁ may have (p+1) folds, then each group of the groups of ML technique(s) 224j_{m,1}, . . . , 224j_{m,n} in the set of ML technique(s) 224jₘ includes (p+1) ML technique(s), each of which is trained on different ones of a plurality of CD labelled dataset folds 210j_{1,1}, . . . , 210j_{1,p} and 210j_{1,All} corresponding to said each set of CD labelled dataset folds 210j₁. This is performed for each of the sets of CD labelled dataset folds 210j₁, . . . , 210jₙ, which results in a set of trained models 225jₘ comprising the n groups of trained models 225j_{m,1}, . . . , 225j_{m,n}, in which each group of trained models 225j_{m,1}, . . . , 225j_{m,n} includes multiple trained models (e.g. a first group of trained models based on ML technique Mm may be represented by Mm_LDSj_D1_F1, Mm_LDSj_D1_F2, . . . , Mm_LDSj_D1_Fp, and Mm_LDSj_D1_All) based on the corresponding sets of CD labelled dataset folds 210j₁, . . . , 210jₙ.

For example, the group of ML techniques 224j_{1,1} (e.g. RF ML technique) is trained on the set of CD labelled datasets 206j₁ (e.g. LDSj_D1), which comprises the set of CD labelled dataset folds 210j_{1,1}, . . . , 210j_{1,p} and 210j_{1,All}. This means that the group of ML techniques 224j_{1,1} includes (p+1) trained ML techniques based on RF ML technique in which each ML technique is configured with same hyperparameters 222a but trained on a different CD labelled dataset fold of the plurality of CD labelled dataset folds 210j_{1,1}, . . . , 210j_{1,p} and 210j_{1,All}. Training the group of ML techniques 224j_{1,1} on the plurality of CD labelled dataset folds 210j_{1,1}, . . . , 210j_{1,p} and 210j_{1,All}, thus generates a corresponding group of trained models 225j_{1,1} (e.g. M1_LDSj_D1_F1, M1_LDSj_D1_F2, . . . , M1_LDSj_D1_Fp, and M1_LDSj_D1_All), which includes (p+1) trained models for the CD labelled dataset folds 210j_{1,1}, . . . , 210j_{1,p} and 210j_{1,All}. Similarly, the group of ML techniques 224j_{1,n} (e.g. RF ML technique) is trained on the set of CD labelled datasets 206jₙ (e.g. LDSj_Dn), which comprises the set of CD labelled dataset folds 210j_{n,1}, . . . , 210j_{n,p} and 210j_{n,All}. This means that the groups of ML techniques 224j_{1,n} are each trained on a corresponding CD labelled dataset fold of the CD labelled dataset folds 210j_{n,1}, . . . , 210j_{n,p} and 210j_{n,All}. This generates the group of trained models 225j_{1,n}, which includes (p+1) trained models each corresponding one of the CD labelled dataset folds 210j_{n,1}, . . . , 210j_{n,p} and 210j_{n,All}.

Each trained model in a group of trained models may be identified by the particular selected set of hyperparameters, a particular dataset, and particular ML technique, the particular folds that were used to train and generate that the trained models in the group of trained models. For example, each model in a group of trained models 225a_{1,1} is based on a group of ML techniques 224a_{1,1} (e.g. group of ML techniques labelled M1) and a CD labelled dataset 206a (e.g. LDSa_D1) that is partitioned into a set of CD labelled dataset folds 210a₁ (e.g. LDSa_D1_F1, LDSa_D1_F2, . . . , LDSa_D1_Fp, and LDSa_D1_All) for a particular selected set of hyperparameters 222a. Each model in the group of trained models 225a_{1,1} may be represented by a unique identifier (e.g. M1_LDSa_D1_F1, M1_LDSa_D1_F2, . . . , M1_LDSa_D1_Fp, and M1_LDSa_D1_All) to enable identification of the parameters, ML technique, and dataset used to generate the model. For example, each model in the group of trained models 225a_{1,1} may be represented by one or more identifier(s) or a combination of identifier(s) indicating at least one or more from the group of: the type of ML technique, the set of hyperparameters, and the group of CD labelled dataset folds (e.g. M1_LDSa_D1_F1, M1_LDSa_D1_F2, . . . , M1_LDSa_D1_Fp, and M1_LDSa_D1_All).

In this manner, in each iteration over the plurality of sets of hyperparameters, the MGT apparatus 224 outputs from each MGT 224a-224j a plurality of trained models 225a-225j. The plurality of trained models 225a-225j have been generated based on each selected set of hyperparameters 222a-222m and the corresponding one or more m ML techniques and datasets 210a-210j. The plurality of trained models 225a-225j includes a plurality of sets of trained model(s) 225a₁, . . . , 225aₘ, . . . , 225j₁, . . . , 225jₘ. Each set of the plurality of sets of trained models 225a₁, . . . , 225aₘ, . . . , 225j₁, . . . , 225jₘ includes a number of n groups of trained model(s). For example, the set of trained models 225a₁ includes the groups of trained models 225a_{1,1}, . . . , 225a_{1,n}, and the set of trained models 225jₘ includes the groups of trained models 225j_{m,1}, . . . , 225_{jm,n}. Each group of trained models includes (p+1) trained models each corresponding one of the CD labelled dataset folds of a set of CD labelled datasets.

For example, the group of trained models 225a_{1,1}, which includes (p+1) trained models based on the 1-st type of ML technique (e.g. RF ML technique) trained on each of a corresponding ones of the CD labelled dataset folds 210a_{1,1}, . . . , 210a_{1,p}, and 210a_{1,All}. The group of trained models 225j_{1,n}, which includes (p+1) trained models based on the 1-st type of ML technique (e.g. RF ML technique) trained on each of a corresponding ones of the CD labelled dataset folds 210j_{1,1}, . . . , 210j_{1,p} and 210j_{1,All}. The group of trained models 225a_{1,n}, which includes (p+1) trained models based on the 1-st type of ML technique (e.g. RF ML technique) trained on each of a corresponding ones of the CD labelled dataset folds 210a_{n,1}, . . . , 210a_{n,p} and 210a_{n,All}. The group of trained models 225j_{m,n}, which includes (p+1) trained models based on the m-th type of ML technique trained on each of a corresponding ones of the CD labelled dataset folds 210j_{n,1}, . . . , 210j_{n,p} and 210j_{n,All}.

The MGT 224 in each iteration outputs a plurality of sets of trained models 225a-225j for each selected set of hyperparameters 222a-222m from a number Hof a plurality of sets of hyperparameters 222 for H>>1, for each of the corresponding one or more of a number of M ML techniques for M>=1, and for each of a number of J sets of CD labelled datasets 210a-210j for J>=1, which includes a number of J·n·(P+1) of a plurality of CD labelled dataset folds. The plurality of sets of trained models 225a-225j includes a plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}. For example, the set of trained models 225a includes the groups of trained models 225a_{1,1}, . . . , 225a_{1,n}, . . . , 225a_{m,1}, . . . , 225a_{m,n}, and so on, and the set of trained models 225j includes the groups of trained models 225j_{1,1}, . . . , 225j_{1,n}, . . . , 225j_{m,1}, . . . , 225j_{m,n}. Each group of trained models corresponding to a set of (P+1) CD labelled dataset folds. The MGT 224 for each iteration outputs a number of J·n·M of a plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}.

The plurality of sets of trained models 225a-225j are received by a corresponding set of model statistics calculation (MSC) apparatus, which in this example includes MSCs 226a-226j for each of the plurality of sets of trained models 225a-225j. Each MSC 226a-226j is configured for calculating the MPSs of the corresponding sets of trained models 225a-225j. For the plurality of trained models 225a-225j, each MSC 226a-226j calculates MPSs based on the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}, for each trained model based on each fold of the set of dataset folds corresponding to each dataset; and storing data representative of the trained model in a set of optimal models based on the calculated MPSs.

The MPSs of a trained model may comprise or represent an indication or a measure of the accuracy and/or performance of the trained model. The MPSs calculated for each trained model may be based on, by way of example only but is not limited to, one or more from the group of: positive predictive value or precision of the trained model; sensitivity, true predictive rate, or recall of the trained model; a receiver operating characteristic, ROC, graph associated with the trained model; an area under a precision and/or recall ROC curve associated with the trained model; any other function associated with precision and/or recall of the trained model; and any other MPS(s) for evaluating the accuracy or performance of each of the trained models. MPSs may be based on the category of ML technique used. For example, if the ML technique used to train and generate a trained model is classification based, then the MPSs that may be used may include or be based on, by way of example only but is not limited to, area under the curve (AUC), area under the precision recall curve (AUprC), F1 score, precision, recall, accuracy, sensitivity, and/or specificity and the like. If the ML technique used to train and generate a trained model is regression based, then the MPSs that may be used may include or be based on, by way of example only but is not limited to, r2 (r squared), root mean squared error (RMSE), mean squared error (MSE), median absolute error, mean absolute error and the like. It is to be appreciated that for any other category of ML technique used to train and generate a trained model, then the MPS that may be used may be based on one or more of the suitable MPSs associated with assessing, by way of example only but is not limited to, the performance and/or accuracy of the trained model based on that category of ML technique.

In this example, calculating the MPSs for each trained model is based on cross-validating each of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}. Cross-validating each of the plurality of the groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} required generating multiple groups of CD labelled dataset folds 210a₁, . . . , 210aₙ, . . . , 210j₁, . . . , 210jₙ for each of the plurality of sets of CD labelled datasets 206a₁, . . . , 206aₙ, . . . , 206j₁, . . . , 206jₙ. This included training each of the m ML techniques to generate said each model of the plurality of groups of models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} on each of the multiple groups of folds 210a₁, . . . , 210aₙ, . . . , 210j₁, . . . , 210jₙ.

MSC apparatus 226a-226j may be used to generate MPS(s) for each trained model of the groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}. This may be achieved by calculating MPS(s) for each trained model in a group and combining the MPSs of each other trained model in the corresponding fold or group to generate a MPS for that group of trained models. Each group of trained models may be identified by the particular selected set of hyperparameters, a particular dataset, and particular ML technique that was trained to generate that group of trained models. The MPSs for each group of models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} is may be used for assessing the cross-validation performance of each of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} to enable selection of the topmost performing models for storing as a set of “optimal” trained models.

The MS apparatus 226a-226j may calculate the MPSs for each of the plurality of sets of trained models 226a-226j or each of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}. Alternatively or additionally, the MPSs for each of the trained models 226a-226j may have been calculated during the generation of the trained models 226a-226j and output by MGT 224 to MS 226a-226j, which may collate and combine the calculated MPSs for each group of models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} for assessment.

In this example, the MS 226a-226j calculates MPSs based on the folds of each group of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}. For example, MS 226a may include a set of MS 226a₁-226aₘ that are used to calculate MPSs on the folds of each corresponding groups of trained models 225a_{1,1}, . . . , 225a_{1,n}, . . . , 225a_{m,1}, . . . , 225a_{m,n}. MS 226a₁ calculates MPSs for the groups of trained models 225a_{1,1}, . . . , 225a_{1,n}, and so on, and MS 226aₘ calculates MPSs for the groups of trained models 225a_{m,1}, . . . , 225a_{m,n}. Similarly, MS 226j may include a set of MS 226j₁-226jₘ that are used to calculate MPSs on the folds of each corresponding groups of trained models 225j_{1,1}, . . . , 225j_{1,n}, . . . , 225j_{m,1}, . . . , 225j_{m,n}. MS 226j₁ calculates MPSs for the groups of trained models 225j_{1,1}, . . . , 225j_{1,n}, and so on, and MS 226jₘ calculates MPSs for the groups of trained models 225j_{m,1}, . . . , 225j_{m,n}.

For example, a MPS calculation for the group of trained models 225a_{1,1} is performed by MS 226a₁. The group of trained models 225a_{1,1} includes (P+1) trained models that have been trained based on a (P+1) CD labelled dataset folds 210a_{1,1}, . . . , 210a_{1,p} and 210a_{1,All}. This produces P trained models based on CD labelled dataset folds 210a_{1,1}, . . . 210a_{1,p} each of which are a different partition or portion of the CD labelled dataset 206a₁ and a trained model based on CD labelled dataset fold 210a_{1,All}, which is the entire CD labelled dataset 206a₁. Cross-validation is performed for the trained models trained on CD labelled dataset folds 210a_{1,1}, . . . , 210a_{1,p} to yield MPSs for each of these trained models. A set of MPSs is calculated based on calculating the MPSs for each of the models trained on CD labelled dataset folds 210a_{1,1}, . . . 210a_{1,p} (e.g. Precision and Recall or Area under Precision Recall Curves etc.). The set of MPSs is combined (e.g. weighted combination or other combination) to form an estimate of the MPSs for the trained model trained on CD labelled dataset fold 210a_{1,All}. The MPS estimate for the trained model trained on CD labelled dataset fold 210a_{1,All} becomes the MPS for the group of trained models 225a_{1,1}. The MPS calculation is performed for each group of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}. This results in a MPS estimate for each group of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}.

The MPS estimates of each group of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} are sent from MS 226a-226j to trained model assessor (TMA) apparatus, which in this example include TMAs 228a-228j for each of the plurality of sets of trained models 225a-225j. The TMAs 228a-228j are configured for selecting from the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} and storing the best performing trained models in a model database 232. For example, the TMAs 228a-228j may select one or more groups of trained models from the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} based on whether MPS estimates calculated for each group by MSs 226a-226j and/or MGTs 224a-224j meet an MPS threshold or that meet one or more MPS criteria or conditions that may be used to select the best trained models. The selected trained models may be stored in model database 232 in a set of optimal trained model(s). The trained models in the set of optimal trained models are optimal in the sense that each of these trained models meet a particular set of MPS threshold(s), condition(s) or criteria(ion). For example, the MPS estimates of each trained model suitable for inclusion to the set of optimal trained models may be greater than or equal to one or more predetermined MPS threshold(s).

Data representative of a trained model and the MPS of the trained model may be stored in the model database 232. Storing a trained model associated with a group of trained models in the model database 232 may include storing data representative of the trained model or group of trained models such as, by way of example only but is not limited to, data representative of one or more, or a combination of: the identity of the trained model or an identifier for the trained model; an indication of the ML technique use to generate the trained model; data representative of the trained model such as, by way of example only but not limited to, weights, coefficients and/or parameters or other data defining the structure of the model; the calculated MPS estimate(s) of the trained model; an indication or identity of the CD labelled dataset used for training the ML technique that generated the trained model; the set of hyperparameters associated with configuring the ML technique that generated the trained model; any other indications or parameters that are useful for storing and using the trained model; and/or the necessary data or information required for training and generating the trained model.

For example, if, during an iteration over the plurality of sets of hyperparameters 222, the trained model that is selected for storage in the model database 232 was the group of trained models 225a_{1,1}, then data representative of the group of trained models 225a_{1,1} may include, by way of example only but is not limited to, data representative of: the group of ML techniques 224a_{1,1} or type of ML technique used to generate the group of trained models 225a_{1,1} (e.g. M1 an RF ML technique), an identifier of the group of trained models 225a_{1,1} (e.g. Model_1), the CD labelled dataset 206a₁ or CD labelled dataset folds 210a₁ used to train the group of trained models 225a_{1,1}, and set of hyperparameters 222a used to configured the ML techniques 224a_{1,1} that generated the group of trained models 225a_{1,1}.

The MPS estimates for each group of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} are evaluated to determine whether data representative of a group of trained models 225a_{1,1} may be stored in model database 232 as, by way of example only but not limited to, the set of optimal trained models. For example, as described above, the MPS estimate for the group of trained models 225a_{1,1} may be compared with an MPS threshold. If the MPS estimate for the group of trained models 225a_{1,1} is less than the MPS threshold or does not reach the MPS threshold, then the group of trained models 225a_{1,1} is not included in the set of optimal trained models. The group of trained models 225a_{1,1} may then be deleted or removed from future consideration. However, if the MPS estimate for the group of trained models 225a_{1,1} is greater than or equal to the MPS threshold, then the group of trained models 225a_{1,1} may be, at least in part, included in the set of optimal trained models. For example, data representative of the group of trained models 225a_{1,1} based on the trained model that was trained on the CD labelled dataset fold 210a_{1,All} may be stored in the model database 232 in the set of optimal models. In another example, data representative of each group of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} may be stored in the set of optimal models based on comparing the calculated MPS estimate of each group of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} with one or more thresholds associated with the MPSs.

Alternatively or additionally, data representative of the group of trained models 225a_{1,1} may be stored in the set of optimal models based on comparing the calculated MPS estimate for the group of trained models 225a_{1,1} with the calculated MPS estimates of previously stored trained models in the set of optimal models. If the calculated MPS estimate for the group of trained models 225a_{1,1} is an improvement over or is greater than or equal to the calculated MPS estimates of previously stored trained models in the set of optimal models, then the group of trained models 225a_{1,1} may be stored in the set of optimal models. However, a previously stored trained model from the set of optimal models may be deleted based on the calculated MPS estimates when a trained model of the same model type or based on the same type of ML technique is found to be an improvement over the previously stored trained model. This may be performed for all of the groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n}.

For example, data representative of a group of trained models 225a_{1,1}, which is based on a model of type M1 (e.g. in this example M1 is a RF ML technique) and trained on the set of CD labelled datasets 206a₁, is stored in the optimal set. If a group of trained models 225j_{m,n}, which is based on a model of type Mm and trained on the set of CD labelled datasets 206jₙ, has an MPS estimate that is greater than the MPS estimate of the group of trained models 225a_{1,1}, then data representative of the a group of trained models 225j_{m,n} is stored in the set of optimal models. This is because the model types of the group of trained models 225a_{1,1} and the group of trained models 225j_{m,n} are different, i.e. M1 is a different model/ML technique type to Mm. However, if the group of trained models 225j_{1,n} has an MPS estimate that is greater than the MPS estimate of the group of trained models 225a_{1,1}, then data representative of the group of trained models 225j_{1,n} is stored, whilst the data representative of the stored group of trained models 225a_{1,1} is deleted from the set of optimal trained models. Thus, only the best trained models of a particular model type or type of ML technique from the groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} are stored in the optimal set of trained models, ring data representative of the trained model further comprises storing data representative of the trained model, the calculated model statistics of the trained model, and/or the dataset associated with training the trained model.

Alternatively or additionally, data representative of the group of trained models 225a_{1,1} may be stored in the set of optimal models based on comparing the calculated MPS estimate for the group of trained models 225a_{1,1} with the calculated MPS estimates of previously stored trained models in the set of optimal models. If the calculated MPS estimate for the group of trained models 225a_{1,1} is an improvement over or is greater than or equal to the calculated MPS estimates of previously stored trained models in the set of optimal models, then the group of trained models 225a_{1,1} may be stored in the set of optimal models. However, a previously stored trained model from the set of optimal models may be deleted based on the calculated MPS estimates when a trained model of the same model type (or same type of ML technique) and trained on labelled datasets based on same CD is found to be an improvement over a previously stored trained model.

In another example, data representative of a group of trained models 225a_{1,1}, which is based on a model of type M1 (e.g. in this example M1 is a RF ML technique) and trained on the set of CD labelled datasets 206a₁, is stored in the optimal set. If a group of trained models 225j_{m,n}, which is based on a model of type Mm and trained on a different set of CD labelled datasets 206jₙ, has an MPS estimate that is greater than the MPS estimate of the group of trained models 225a_{1,1}, then data representative of the group of trained models 225j_{m,n} is stored in the set of optimal models. This is because both: 1) the model types of the group of trained models 225a_{1,1} and the group of trained models 225j_{m,n} are different, i.e. M1 is a different model type to Mm; and 2) the training datasets are based on different CDs 206a₁ and 206jₙ. Similarly, if the group of trained models 225j_{1,n} has an MPS estimate that is greater than the MPS estimate of the group of trained models 225a_{1,1}, then data representative of the group of trained models 225j_{1,n} is stored in the set of optimal models. Although the model types of the group of trained models 225a_{1,1} and the group of trained models 225j_{1,n} are the same, i.e. M1, the training datasets are based on different CDs 206a₁ and 206jₙ. However, if the group of trained models 225j_{1,1} has an MPS estimate that is greater than the MPS estimate of the group of trained models 225a_{1,1}, then data representative of the group of trained models 225j_{1,1} is stored in the set of optimal models whilst the data representative of the stored group of trained models 225a_{1,1} is deleted from the set of optimal trained models. This is because both: 1) the model types of the group of trained models 225a_{1,1} and the group of trained models 225j_{1,1} are the same, i.e. both are of type M1; and 2) the training datasets are based on the same type of CDs 206a₁ and 206j₁. Thus, only the best trained models of a particular model type (or type of ML technique) and trained on a CD labelled dataset of a particular CD from the groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} are stored in the optimal set of trained models.

Additionally or alternatively, the MPS estimates of the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} may be ranked and data representative of the S>1 topmost ranked groups of trained models set of optimal models may be stored in the optimal set of models. Additionally or alternatively, the set of optimal models may be further optimised by ranking the groups of trained models stored in the set of optimal models based on their corresponding MPS estimates, where data representative of the topmost T>1 ranked groups of trained models may be retained whilst data representative of the other groups of models may be deleted from the set of optimal models.

An example iteration of a number of iterations over the plurality of sets of hyperparameters 222 has been described with reference to FIG. 2c. Once the plurality of groups of trained models 225a_{1,1}, . . . , 225a_{1,n}, . . . , 225a_{m,1}, . . . , 225a_{m,n}, . . . , 225j_{1,1}, . . . , 225j_{1,n}, . . . , 225j_{m,1}, . . . , 225j_{m,n} have been assessed by TMAs 228a-228j and any selected groups of trained models stored in the model database 232, e.g. in the set of optimal models, a further iteration of training, generation, assessment and storage of selected trained models may be performed based on another selected set of hyperparameters 222a-222m from the plurality of sets of hyperparameters. Thus, the MGTA apparatus 220 performs another iteration by selecting another unique number of m sets of hyperparameters 222a-222m, different from the previous one or more iterations, in which each correspond to a number m of the one or more ML technique(s) used to generate the trained models for the current iteration. The number of iterations that are performed may be predetermined, or simply based on the number of unique sets of m sets of hyperparameters 222a-222m in the plurality of sets of hyperparameters 222. Once the iterations over the plurality of sets of hyperparameters 222 has terminated there will be one or more or a multiple of trained models stored in model database 232. The trained models stored in model database 232 may be stored as a set of optimal trained models. From this model database 232, one or more ensemble of models may be formed or created, benchmarked and stored in an ensemble model database for retrieval and later use.

FIG. 2f is a schematic illustration of an example ensemble system 238 for forming, benchmarking and storing one or more ensemble models based on the trained models stored in the model database 232. The ensemble system 238 includes an ensemble model creation apparatus 240 for creating one or more ensemble models, an ensemble benchmarking apparatus 250 for benchmarking any created ensemble model(s), and a ensemble model database 260 for storing the benchmarked ensemble model(s) for later use etc. After the one or more or a multiple of trained models have been stored in model database 232 and the iterations described in FIG. 2c have terminated, the ensemble creation apparatus 240 may create or form one or more ensemble models based on the trained models in the model database 232. In this example, the trained models stored in model database 232 may be stored as a set of optimal trained models.

The ensemble creation apparatus (ECA) 240 be configured to perform one or more of the following: in step 242, the ECA 240 may retrieve data representative of multiple trained models and their corresponding MPS estimates based on model type and/or type of chemical or compound descriptor (CD) from the model database 232. In step 244, the ECA 240 may select the best trained model from the retrieved multiple trained models. In step 246, the ECA 240 adds the selected trained model to a newly formed ensemble model and, if any further trained models can be retrieved, repeat step 242 based on a different model type and/or type of CD. Steps 242 to 246 may be repeated a predetermined number of times, a number of times as required by the user or operator input for creating an ensemble model, or until no further trained models can be retrieved from model database 232. The ECA 240 may then proceed to step 248, which may further optimise the newly formed ensemble model, which comprises multiple selected trained models selected based on steps 242-246. Step 248 may include pruning the number of trained models from the ensemble model by, by way of example only but is not limited to, removing trained models from the ensemble model that have MPS estimates or accuracy less than a predetermined threshold. In step 249, each of the remaining models (e.g. the models that are not pruned) may be assigned a weight based on, by way of example only but is not limited to, the accuracy and/or MPS estimates of each model. For example, each model may be assigned a weight that is proportional to the accuracy or MPS estimate of that model. When used in an ensemble model, this weight may be applied to the output of the model to adjust its influence on the ensemble model output. In another example, weights may be assigned to the models in such a manner that the most accurate models (or models with best MPS estimates) in an ensemble have more influence over less accurate models (or models with a lower MPS estimates) in the ensemble. It is to be noted that step 249 may be optional. Once an ensemble model has been created by ECA 240, the ensemble benchmark apparatus (EBA) 250 benchmarks the created ensemble model and determines whether to store the ensemble model as a final ensemble model in ensemble model database 260.

In steps 242 and 244, the ECA 240 may retrieve multiple models and select the best trained model from the retrieved multiple trained models. This may include, by way of example only but is not limited to, selecting a subset of optimal trained models from the set of optimal trained model(s) in the model database 232, where each trained model in the subset of optimal trained models has improved MPS estimates compared with the remaining trained models in the set of optimal trained models. As another example, selecting the subset of optimal models from the set of optimal model(s) may further include ranking the optimal models based on the MPS estimates and/or accuracy etc., and selecting a subset of the topmost S ranked optimal models, S>=number of models required in the ensemble model or 2, for inclusion into the ensemble model.

Alternatively or additionally, steps 242 and 244 may include one or more of the following: selecting a subset of optimal models from the set of optimal model(s) by retrieving models and associated MPS estimates (or model statistics) from the set of optimal trained models that correspond to the same model type (or type of ML used to train the trained models), and/or same CD; ranking the retrieved models based on the MPS estimates; and selecting one or more trained model(s) from the retrieved trained models having the highest MPS estimates for inclusion into the ensemble model. Alternatively or additionally, steps 242 and 244 may further include: for each of the plurality of CD labelled datasets 206a₁, . . . , 206aₙ, . . . , 206j₁, . . . 206jₙ: retrieving the trained models and associated MPS estimate(s) and/or accuracy from the set of optimal trained models that are associated with the same CD labelled dataset; ranking the retrieved trained models based on the MPS estimates or any other model statistics; and selecting one or more topmost model(s) from the ranked retrieved models for inclusion into the ensemble model.

Once the ensemble model has been formed, further ensemble models may be created based on steps 242-248. For example, one or more further ensemble models may be created or formed based on different combinations of model type(s) and/or CD(s), which may be specified by an operator or user, or automatically and/or randomly generated/selected. In another example, one or more further ensemble models may be created or formed from any remaining trained models in the model database that have not been used in an ensemble model. Once one or more ensemble model(s) have been formed and/or created, the EBA 250 may be used to benchmark one or more ensemble models to assist in determining whether one or more of the ensemble model(s) may be stored in the ensemble database 260.

FIG. 2g is a schematic diagram illustrating an example ensemble benchmark apparatus (EBA) 250 for benchmarking the one or more ensemble models. The EBA 250 is configured to retrieve the models corresponding to each single descriptor CD of the set of CD descriptors and corresponding single dataset fold of the set of CD labelled dataset folds 210a-210j from database 232. In step 252a, the EBA 250 puts together all the models corresponding to a first descriptor CD and a corresponding single dataset fold (e.g. fold F0) into first ensemble. For this single dataset fold (e.g. fold F0), each model is trained on a certain percentage X (e.g. 80%) of the data in that fold. Each model may be trained on a different portion or parts of the single dataset fold (e.g. fold F0). Once trained, each model is tested on the remaining Y=100%−X (e.g. 20%) of the data in that dataset fold to estimate the performance of that model, e.g. an estimate MPS may be generated. Again, the remaining dataset fold for each model may be different, hence each model may be tested on different portion of parts of the remaining dataset fold.

This process is repeated for all other folds of the dataset (e.g. fold F1, fold F2 . . . ) for that particular single descriptor CD. The average of the MPSs across the dataset folds for that particular single descriptor CD, as well as, the MPSs for each individual dataset fold for that particular single descriptor CD are stored in an ensemble database 260 alongside the ensemble model trained on 100% of the dataset folds for that particular single descriptor. The process is further repeated for each different descriptor CD of the set of CD descriptors.

As an example, the EBA 250 perform one or more of the following: in step 252, the EBA 250 may retrieve data representative of the trained models associated with an ensemble model from the model database 232. In steps 252a-252p, the EBA 250 retrieves all the trained models in the ensemble model for a particular single fold from the corresponding set of CD labelled dataset folds 210a-210j. In step 254, the EBA 250 may create or recreate the ensemble model from the retrieved trained models based on a selected fold, which may be selected based on the MPSs associated with the folds. In step 256, the EBA 250 calculated MPS for the created ensemble model by testing against CD labelled test sets for each fold. After this, the MPSs for the created ensemble model are stored along with the ensemble model in ensemble database 260.

Alternatively or additionally, benchmarking the one or more ensemble models may further include calculating ensemble MPSs (or model statistics) based on cross-validating each of the one or more ensemble models.

The ensemble database 260 may be used to retrieve a selected ensemble model for use in a particular application. For example, an ensemble model may be selected for use in modelling, by way of example only but not limited to, a process or a problem associated with compounds, or determining a relationship with an input compound (e.g. an ensemble model may be trained to predict whether a compound has a particular property) and the like. When an ensemble model is selected, it may be already configured for receiving an input dataset and outputting a corresponding result dataset according to the application.

Given that the selected ensemble model includes multiple trained models, each selected from an optimal set of models, the ensemble model may not be optimised on combining outputs from each of the multiple trained models. So-called stacking may be applied to estimate how best to combine the classification/prediction outputs from each of the multiple trained models of an ensemble model when given an input dataset. Stacking typically yields performance better than any single one of the trained models of an ensemble model. Typically, stacking involves training a machine learning (ML) technique (or learning algorithm) to combine the predictions or output data results of the trained models of the ensemble. Initially, the models of the ensemble may be trained using an available labelled training dataset, then a combiner ML technique or algorithm is trained to generate a combiner ML model/algorithm for making a final prediction or the final output data result using all of the predictions or output data results of the trained models as inputs to the combiner ML technique or algorithm. Given that the ensemble model may already include a set of trained models, the initial step of training the models may not be necessary, rather, just the combiner ML model/algorithm may be trained based on the labelled datasets that were used to train the ML models and the like. The choice of the ML technique or algorithm for using in generating the combiner ML model or combiner algorithm may be made based on the demands of the application of the ensemble model. Although a logistic regression ML technique may typically be used, by way of example only but is not limited to, for the combiner algorithm, it is to be appreciated by the skilled person that any arbitrary combiner algorithm or combiner ML technique may be used to train a combiner ML model or algorithm, which means that any type of ensemble model technique may be derived or implemented.

Although stacking has been described above, by way of example only but is not limited to, when an ensemble model is retrieved from the ensemble database, it is to be appreciated by the skilled person that stacking and generation of the combiner ML model/technique may be implemented at any stage after the ensemble model has been created. For example, as described with respect to FIGS. 2f and 2g, stacking for each ensemble model may be applied when an ensemble model is created by ensemble system 238, which includes an ensemble model creation apparatus 240 that implements an ensemble creation process for creating one or more ensemble models. The resulting ensemble model, which may include a trained combiner ML technique/model may be stored in the ensemble model database. Similarly, as described with respect to FIGS. 2f and 2g, an ensemble benchmarking apparatus 250 for implementing a benchmarking process for benchmarking any created ensemble model(s) may also include a stacking process prior to, during, or after the benchmarking process in which the benchmarked ensemble model, include the trained combiner ML model/algorithm from the stacking, may be stored in the ensemble model database 260 for later use etc. When the ensemble model is stacked may depend on the time taken for stacking an ensemble model, thus, the skilled person may appreciate that it may be applied at any time. Furthermore, the combiner ML model(s) of any of the ensemble models for which stacking has been applied and stored in the ensemble database 260 may need to be updated from time-to-time, thus, these ensemble model(s) may be retrieved and re-stacked in which the combiner ML model/algorithm is replaced by an updated or different combiner ML model/algorithm.

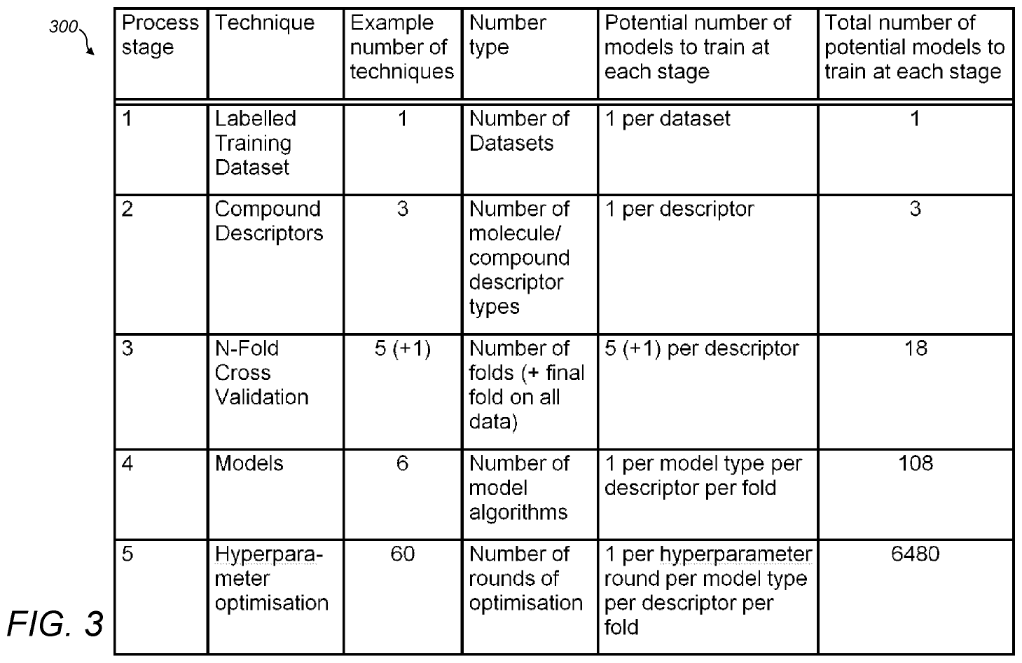

FIG. 3 is a table 300 illustrating a small scale example of the complexity of training, generating and assessing a plurality of models for use in an ensemble according to the invention. This example illustrates that the total number of models that can be trained and evaluated is beyond a manual exercise all but the simplest cases. In fact, the number of models typically increases in an exponential-like manner due to the numbers of different variables such as, by way of example only but not limited to, the training dataset(s), the compound descriptors (CDs), the type(s) of model(s), each set of hyperparameter(s) requiring optimisation over each model, and the N-fold cross-validation performed on each model.

In the first process stage and as described with respect to FIG. 2a, a number of labelled training datasets 202a-202j may be selected for training one or more models associated with the same objective or prediction type. In this example, only one training dataset 202a is selected for training the models. It is to be appreciated that more than one dataset may be selected for training the models. In the second process stage, a number n of CDs (also known as molecule descriptor types) are selected, which in this example is 3. Thus, the labelled training dataset 202a is duplicated 3 times in which each labelled training dataset uses a different CD of the 3 selected CDs. Thus, a set of CD labelled datasets 206a may be generated, where the set of CD labelled datasets 206a include 3 different CD labelled datasets.

In process stage 3, P-fold cross validation may be performed for each model and each dataset, thus each labelled CD dataset in the set of CD labelled datasets is partitioned into P different folds plus a final fold including the all the dataset. In this case, P=5 such that the number of folds is 5 (+1 fold on all the data) to generate a set of CD labelled dataset folds for each of the 3 CDs. In this case, there are 18 CD labelled dataset folds. FIG. 2c illustrates the hyperparameter optimisation and selection of optimal models for storage in database 232 where a number of m types of models are selected for generation/evaluation on each CD labelled dataset fold. In this example, at process stage 4, a number of 6 model types are selected for generation/evaluation. Thus, each CD labelled dataset fold will be used to generate 6 different models for evaluation. Currently, without hyperparameter optimisation, a total of 108 different models will be generated and evaluated for selecting those models with the best MPSs. However, with hyperparameter optimisation, then further models may be optimised for each different set of hyperparameters. Thus, there is one model to be trained per hyperparameter set/round per model type per descriptor per fold. In this example, for simplicity, when there are 60 sets of hyperparameters, i.e. 60 rounds of hyperparameter optimisation, the total number of models that may be trained/generated is 6480.

The ensemble model optimisation and generation according to the invention and/or based on the method(s), process(es), system(s) and/or apparatus as described herein with reference to FIGS. 1a-3a is configured to generate and select from a large number of trained models, or a plurality of sets of trained models, with the same or similar objectives a subset of the best performing trained models that can be used to create one or more ensemble model(s) that have been optimised for modelling a process or problem associated with one or more compounds. The trained models are based on one or more ML technique(s) or a plurality of ML technique(s) and corresponding plurality of sets of hyperparameters, one or more labelled datasets and/or dataset folds generated for each compound descriptor in a set of compound descriptors. The trained models are assessed based on MPSs of the models and the best performing trained models selected and stored for forming the one or more ensemble model(s).

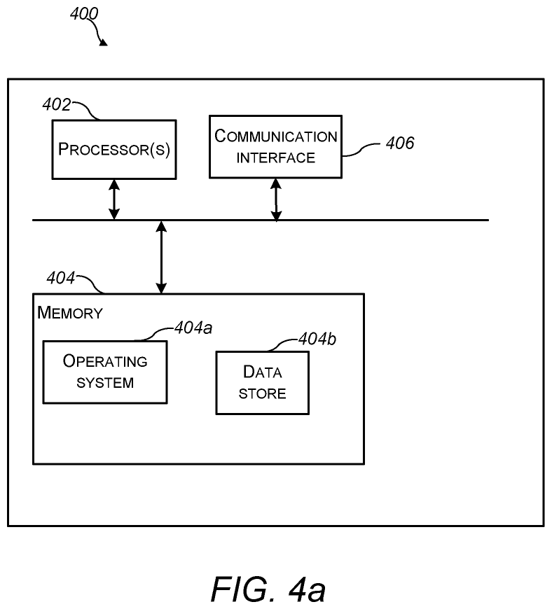

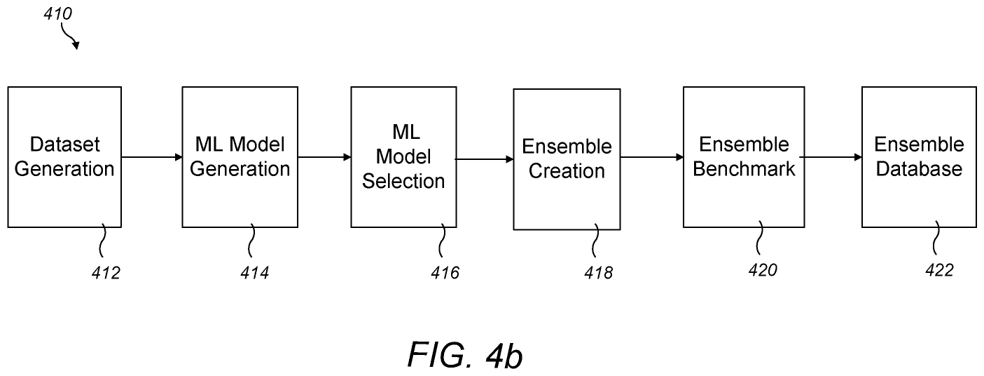

FIG. 4a is a schematic diagram illustrating a example computing device 400 that may be used to implement one or more aspects of the ensemble model generation according to the invention and/or includes the methods and/or system(s) and apparatus as described with reference to FIGS. 1a-3, 4b to 5d. Computing device 400 includes one or more processor unit(s) 402, memory unit 404 and communication interface 406 in which the one or more processor unit(s) 402 are connected to the memory unit 404 and the communication interface 406. The communications interface 406 may connect the computing device 400 with one or more databases or other processing system(s) or computing device(s). The memory unit 404 may store one or more program instructions, code or components such as, by way of example only but not limited to, an operating system 404a for operating computing device 400 and a data store 404b for storing additional data and/or further program instructions, code and/or components associated with implementing the functionality and/or one or more function(s) or functionality associated with generating and/or using CD labelled datasets and/or CD labelled dataset folds and the like, training, generation, and assessing a plurality of model(s), selecting and storing one or more trained models in a model database, creating or forming an ensemble model based on the stored trained models, one or more of the method(s) and/or process(es) of the apparatus and/or system(s)/platforms as described with reference to at least one of FIGS. 1a to 3, 4b to 5d.

Further aspects of the invention may include one or more apparatus, systems and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es) or combinations thereof as described herein with reference to FIGS. 1a to 3, 4b to 5d.

Other aspects of the invention may include an apparatus including a processor and a memory unit, the processor is connected to the memory unit, where: the processor is configured to train a plurality of models based on a plurality of datasets associated with compounds; the processor is configured to calculate model performance statistics for each of the plurality of trained models; the processor and memory are configured to selecting and storing a set of optimal trained model(s) from the trained models based on the calculated model performance statistics; and the processor and memory are configured to form one or more ensemble models, each ensemble model comprising multiple models from the set of optimal trained model(s).

Further aspects of the invention may include an apparatus including a processor, a memory unit and a communication interface, the processor is connected to the memory unit and the communication interface, where: the processor and communication interface are configured to retrieve an ensemble model generated the process(es) 100, 120, 500 and/or apparatus/systems 200, 220, 238, 250, 400, 410, and/or any method(s)/process(es), step(s) of these process(es), modifications thereof, as described with reference to any one or more FIGS. 1a to 4b and/or the above apparatus, and/or as described herein; the processor and memory are configured to input, to the ensemble model, data representative of one or more labelled dataset(s) used to generate and/or train the model(s) of the ensemble model; and the processor and memory are configured to receive, from the ensemble model, output data associated with labels of the one or more labelled dataset(s).

In another aspect, the invention may include an apparatus including a processor, a memory unit and a communication interface, the processor is connected to the memory unit and the communication interface, where: the processor is configured to input, to an ensemble model for modelling a process or problem associated with compounds, representations of one or more compound(s); the processor and/or memory are configured to receive, from the ensemble model, results associated with modelling the process or problem based on the one or more compound(s); and where the ensemble model comprises multiple model(s) automatically selected based on model performance statistics calculated for each of the model(s).

FIG. 4b is a schematic diagram illustrating of a example ensemble system 410 that may be used to implement one or more aspects of the ensemble model generation according to the invention and/or implementing one or more of the methods and/or system(s) and apparatus as described with reference to FIGS. 1a-3, 4b to 5d. The system 410 for generating an ensemble model includes a dataset generation module or apparatus 412, a model generation module or device 414, a model selection module or device 416, and an ensemble creation module or device 418, which are connected together.

In operation, the dataset generation module 412 is configured for generating a plurality of datasets associated with compounds based on multiple labelled datasets. The generated plurality of datasets are sent to the model generation module 414, which is configured to train a plurality of models based on the generated plurality of datasets associated with compounds. The model generation module 414 may be further configured to calculate model performance statistics are calculated for each of the plurality of trained models. Alternatively or additionally, an model statistics calculation module or device (not shown) may calculated the required model performance statistics. The plurality of trained models and the model performance statistics are sent to the model selection module 416. The model selection model 416 is configured to select and store a set of optimal trained model(s) from the plurality of trained models based on the calculated model performance statistics. Thus, an optimal set of trained model(s) may be formed and stored for use in creating an ensemble model. The ensemble creation module 418 is configured to retrieve multiple models from the set of optimal trained models that have been stored, and forms one or more ensemble models, each ensemble model comprising multiple models from the set of optimal trained model(s). The created ensemble models may be stored for subsequent selection, retrieval and use for predicting and/or classifying input data representative of compounds, typically not seen by the ensemble models during training, in accordance with the model generated based on the labelled datasets used to train the models in each ensemble model.

The system 410 further includes an ensemble benchmark module or device 420 and an ensemble database 422 coupled to the ensemble creation module 418. The ensemble benchmark module 420 may be configured to retrieve from storage one or more of the created/formed ensemble model(s) and perform benchmark tests to determine benchmark results comprising data representative of ensemble model performance statistics for the retrieved ensemble model based on the corresponding plurality of datasets used to generate each of the models forming the retrieve ensemble model. The retrieve ensemble model and the corresponding benchmark results may be sent to the ensemble database module 422 for storing the benchmarked ensemble models and corresponding benchmark results for later selection, retrieval and use.

The system 410 may be further configured to implement the method(s), process(es), apparatus and/or systems as and/or as described herein or as described with reference to any of FIGS. 1a to 5d. For example, a dataset generation module or apparatus 412 may be further configured to implement the functionality, method(s), process(es) and/or apparatus associated with generating the plurality of datasets based on using CD labelled datasets and/or CD labelled dataset folds and the like and/or as described herein or as described with reference to FIGS. 1a, 2a, 2b and/or 4a, modifications thereof and the like. The model generation module or device 414 may be further configured to implement the functionality, method(s), process(es) and/or apparatus associated with training and/or optimising the models in relation to their hyperparameters based on the generated plurality of datasets, calculating model performance statistics in relation to each of the trained models and the like and/or as described herein or as described with reference to FIGS. 1a, 2c to 2e and/or 4a, 4b to 5d. The model selection module or device 416 may be configured to implement the functionality, method(s), process(es) and/or apparatus associated with assessing a plurality of trained model(s), selecting and storing one or more trained models in a model database based on the model performance statistics, in which a set of optimal models may be stored in the model database, and/or as described herein or as described with reference to FIGS. 1a, 2c to 2e and/or 4a to 5d. The ensemble creation module or device 418 may be further configured to implement the functionality, method(s), process(es) and/or apparatus associated with creating or forming an ensemble model based on the stored trained models from the optimal set of models, and/or as described herein or as described with reference to FIG. 1a, 2f and/or 4a to 5d.

The ensemble benchmark module 420 may be further configured to implement the functionality, method(s), process(es) and/or apparatus associated with benchmarking the created ensemble models and the like and/or as described herein or as described with reference to FIGS. 1a, 2g and/or 4a. The ensemble database module 422 may be further configured to implement the for storing the benchmarked ensemble models and corresponding benchmark results for later selection, retrieval and use and/or as described herein or as described with reference to any of FIGS. 1a to 5d.

The ensemble creation module or device 418 may be configured to implement stacking of each of the created ensemble models. The ensemble benchmark module 420 may be configured to implement stacking of each of the ensemble models that are to be, are, or have been benchmarked. The ensemble database module 422 may further be configured to implement stacking of each of the created ensemble models. Furthermore, stacking of each of the ensemble models retrieved from the ensemble database 260 may be performed and the resulting combiner ML algorithm may be stored along with the ensemble model for subsequent use.

Furthermore, the process(es) 100, 120, 500 and/or apparatus/systems 200, 220, 238, 250, 400, 410, 500, 520, 540, 560 and/or any method(s)/process(es), step(s) of these process(es), modifications thereof, as described with reference to any one or more FIGS. 1a to 5d may be implemented in hardware and/or software. For example, the method(s) and/or process(es) for generating, training and/or implementing an ensemble model and/or for using an ensemble model as described with reference to one or more of FIGS. 1a-5d may be implemented in hardware and/or software such as, by way of example only but not limited to, as a computer-implemented method by one or more processor(s)/processor unit(s) or as the application demands. Such apparatus, system(s), process(es) and/or method(s) may be used to generate an ensemble model including data representative of a set of ML models generated from one or more ML techniques as described with respect to the process(es) 100, 120, 200, 220, 238, 250, 500, 520, 540, 560 and/or apparatus/systems 200, 220, 238, 250, 400, 410, 500, 520, 540, 560 and/or any method(s)/process(es), step(s) of these process(es), modifications thereof, as described with reference to any one or more FIGS. 1a to 5d, modifications thereof, and/or as described herein and the like. Thus, a an ensemble model may be obtained from computer-implemented method(s), process(es), method(s) 100, 120, 200, 220, 238, 250, 500, 520, 540, 560 and/or apparatus/systems 200, 220, 238, 250, 400, 410, 500, 520, 540, 560 and/or any method(s)/process(es), step(s) of these process(es), modifications thereof, as described with reference to any one or more FIGS. 1a to 5d and/or as described herein.

Furthermore, an ensemble model or a set of models may also be obtained process(es) 100, 120, 200, 220, 238, 250, 500, 520, 540, 560 and/or apparatus/systems 200, 220, 238, 250, 400, 410, 500, 520, 540, 560 and/or any method(s)/process(es), step(s) of these process(es), as described with reference to any one or more FIGS. 1a to 5d, modifications thereof, and/or as described herein, some of which may be implemented in hardware and/or software such as, by way of example only but not limited to, a computer-implemented method that may be executed on a processor or processor unit or as the application demands. In another example, a computer-readable medium may include data or instruction code representative of an ensemble model according to any one of the ensemble model(s) as described above and/or as described herein, which when executed on a processor, causes the processor to implement the ensemble model.

In the embodiment(s) described above the computing device, apparatus and/or systems may be implemented on a server comprising a single server or network of servers. In some examples the functionality of the server may be provided by a network of servers distributed across a geographical area, such as a worldwide distributed network of servers, and a user may be connected to an appropriate one of the network of servers based upon a user location.

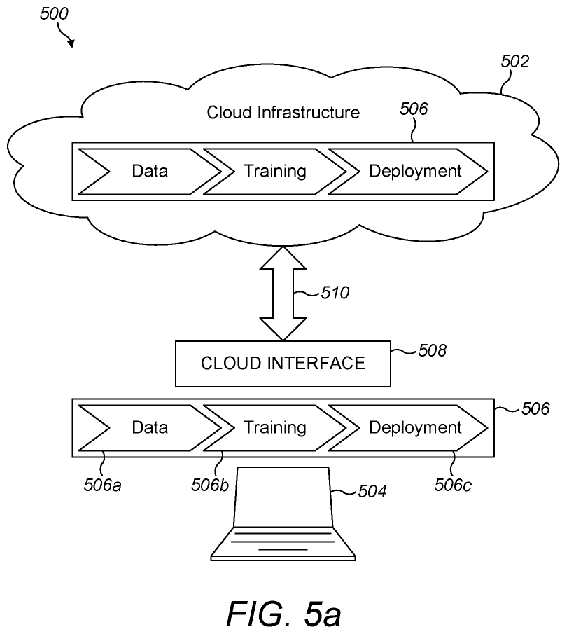

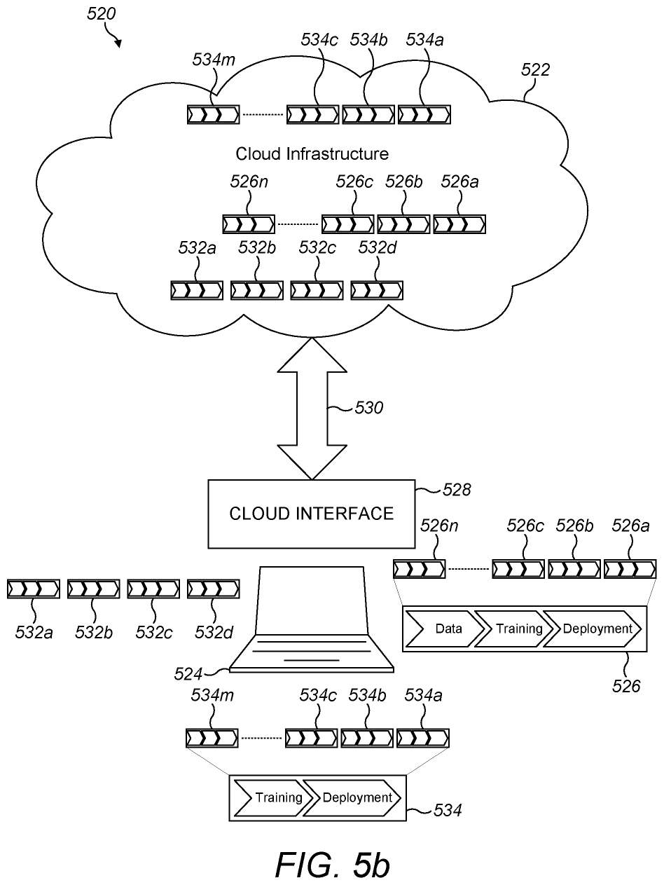

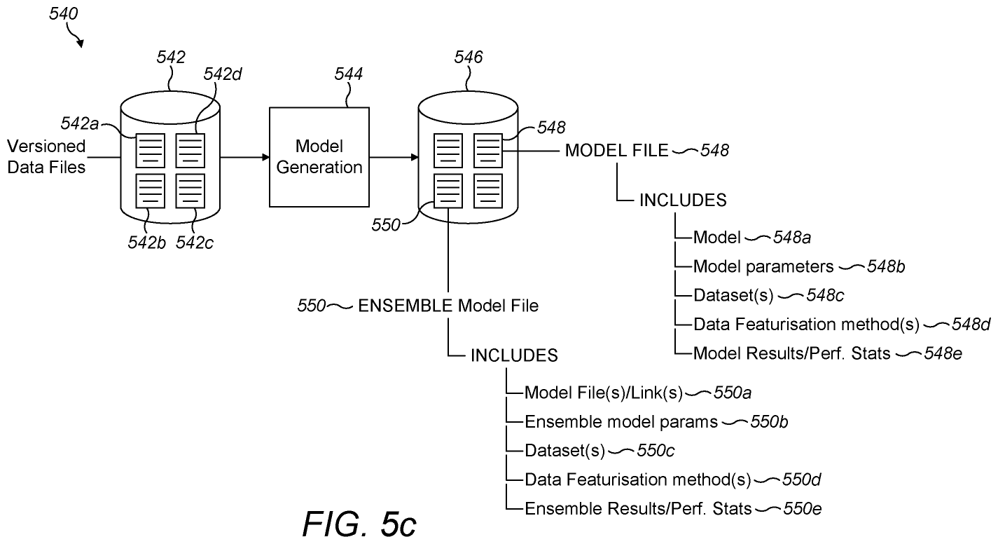

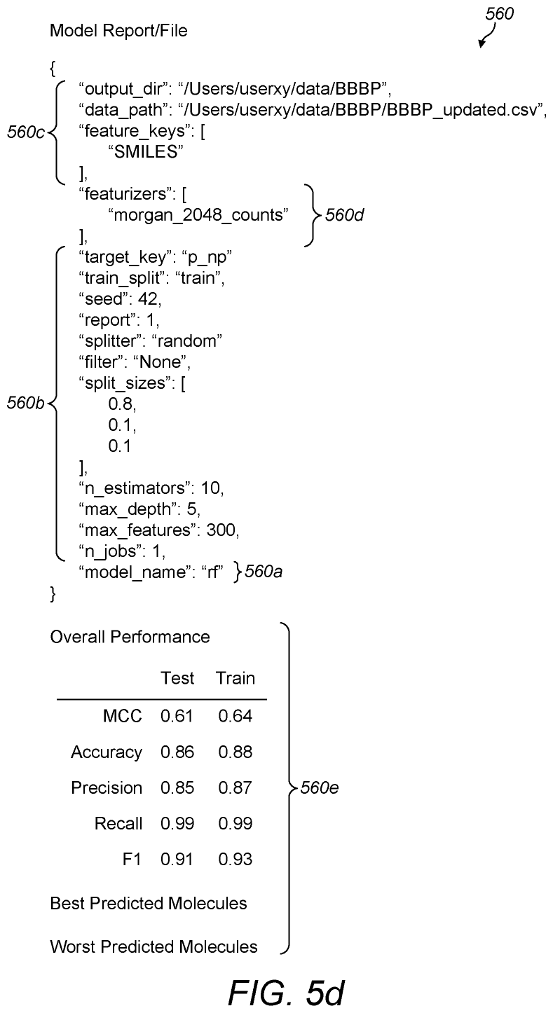

FIG. 5a is a schematic diagram of an example cloud-based system 500 for generating and/or deploying an ensemble model according to the invention or as herein described. The cloud-based system 500 includes a cloud computing infrastructure 502 for generating one or more ensemble models and/or for deploying one or more ensemble models. The cloud computing infrastructure 502 may include a plurality of servers such as, by way of example only but not limited to, a cloud of servers, cluster of servers, and/or a network of servers or computing devices and the like. The plurality of servers may operate on computing tasks or jobs, which are based on executable code and may also include data or references to data on which the executable code may operate. For example, a model training task or job may include executable code associated with, by way of example only but not limited to, model training engine, ML technique for training the model, collecting/assessing results and the like; and data including, by way of example but not limited to, input dataset such as a labelled training dataset for training the model, hyperparameters, performance criteria and the like.

The plurality of servers may be dedicated to processing, after receiving from a user of a computing device 504, one or more ensemble generation/modelling tasks or jobs 506, which are specified by a user of computing device 504. An ensemble generation/modelling task or job 506 may be defined by a user of computing device 504 for generating an ensemble model or for deploying an ensemble model for modelling a particular problem or process and the like or as the application demands. For the ensemble generation task or job 506, the user may specify data representative of: 1) the input dataset 506a; and 2) a plurality of models for training 506b. For the ensemble modelling task or job 506, in which the ensemble model has been generated and is based on multiple trained models, the user may specify data representative of: 1) the input dataset 506a; and 2) the ensemble model or trained models for deployment 506c.

For the ensemble generation task or job 506, the input dataset 506a may be specified and generated as described with reference to FIGS. 2a and 2b. The plurality of models for training 506b may be specified and/or generated/trained as described with reference to FIGS. 2c and 2d, where the input dataset 506a and sets of hyperparameters are used to train a set of models based on the specified plurality of models, the set of trained models are assessed in which the best performing trained models are selected for subsequent deployment. The best performing trained models are selected for storage and/or for generating the ensemble model or other ensemble models. The cloud interface 508 (e.g. a REST API) may receive the ensemble generation task or job 506 from computing device 504 and package and send, via a communications network 510, the entire ensemble generation task or job 506 to the cloud computing infrastructure 502 for processing and generating the ensemble model as described with reference to FIGS. 1a to 2g. As can be seen, the ensemble generation task or job 506 is processed by the cloud computing infrastructure 502 as one large task or job 506 in which the results, which are a set of trained models are stored in a database, which may include a file system storing trained model files or file objects and the like.

For example, a user of the computing device 504 may specify a selection of chemical or compound descriptors for generating the input dataset 506a as described with reference to FIGS. 2a to 2b for use in training a plurality of models 506b. The user of the computing device 504 may also specify one or more datasets that may be useful for modelling a particular process, problem and/or having a similar objective in the cheminformatics and/or bioinformatics fields. The input dataset 506a includes a plurality of input datasets based on replicating each of the specified datasets in which the chemical or compound descriptors of that dataset are replaced with one of the specified selection of chemical or compound descriptors. This produces a plurality of input datasets representing the same training data but in which each input dataset uses a different chemical or compound descriptor from the specified set of compound descriptors. The user of the computing device 504 may also specify the types of models that are to be trained based on the plurality of datasets along with ranges or sets of hyperparameters for each type of model as described with reference to FIGS. 2c to 2d. These may be used by the ensemble generation task or job 506 in jointly iterating/searching over the combination of chemical or compound descriptor input datasets and sets of hyperparameters to identify the best performing trained models associated with modelling the particular process, problem and/or having a similar objective in the cheminformatics and/or bioinformatics fields.

The ensemble generation task or job 506 may provide a set of trained models (so-called “optimal” trained models), which may be used to form an ensemble model. The set of trained models are “optimal” in the sense that they are determined to be the best performing trained models that meet certain performance criteria (e.g. model performance statistics and the like) and/or as described with reference to FIGS. 2f and 2g. These models are referred to herein as an “optimal” trained model, which are optimal in the sense that the model performance statistics and the like of the trained model has met certain predefined performance criteria or thresholds as described with reference to FIGS. 2a-2g; the term “optimal trained model” will be used to refer to such trained models. As described with reference to FIGS. 2c to 2g, the set of optimal trained models may be used to generate of form the ensemble model and/or each of the set of optimal trained models may be stored in a database or file structure for later selection for an ensemble model.

For example, the data representative of each optimal trained model and/or each ensemble model that is formed or generated may be stored in a database or record system and the like for later retrieval and/or deployment. The database may be based on a file system that includes, by way fo example only but is not limited to, a set of trained model files or file objects, or a ensemble model files or file objects and the like. As can be seen, the plurality of servers or cluster of servers of the cloud infrastructure is dedicated to running the entire ensemble generation task or job 506 until it has finished processing. That is, until it has finished iterating over all combinations of input datasets 506a, training models and sets of hyperparameters 506b and has found a set of optimal trained models, which may be stored in a database such as a file system as a set of trained model files or file objects, or a ensemble model files or file objects and the like.

FIG. 5b is a schematic diagram of another example cloud-based system 520 for generating and/or deploying an ensemble model according to the invention or as herein described. The cloud-based system 520 includes a cloud computing infrastructure 522 for generating one or more ensemble models. The cloud computing infrastructure 522 may include a plurality of servers such as, by way of example only but not limited to, a cloud of servers, cluster of servers, and/or a network of servers or computing devices and the like. The plurality of servers of the cloud computing infrastructure 522 may be configured to provide a dynamic allocation of computing resources. In a similar manner as for the cloud-based system 500 of FIG. 5a, a user of the computing device 524 may specify 1) the input dataset 506a that may include a plurality of datasets; 2) the plurality of models for training 506b; and/or 3) deployment of trained models and/or ensemble models. This may be used to generate and/or configure, by way of example only but is not limited to, an ensemble model generation task or job 526, one or more model training tasks or job 532a-532b, one or more modelling tasks or jobs 532c-532d, which are based on trained models, an ensemble model deployment task or job 534 and the like or as the application demands.

The computing device 524 and/or cloud interface 528 (e.g. a Python API) may divide or split any large tasks or jobs, such as the ensemble generation task or job 526 into a plurality of model training tasks or jobs 526a, 526b, 526c, to 526n for submission to the cloud computing infrastructure 522. By submitting a plurality of model training tasks or jobs 526a, 526b, 526c, to 526n, the cloud computing infrastructure may more efficiently allocate computing resources of the plurality of servers to processing the plurality of model training tasks or jobs 526a, 526b, 526c, to 526n. The computing device 524 and/or cloud interface 528 (e.g. a Python API) may divide or split any other tasks or jobs, such as the one or more model training tasks or jobs 532a-532b, for training individual models based on input datasets and the like for solving or modelling a particular problem or process and the like or as the application demands. The cloud computing infrastructure may more efficiently allocate computing resources of the plurality of servers to processing the plurality of model training tasks or jobs 532a-532b. Similarly, any of the one or more modelling tasks or jobs 532c-532d, ensemble model deployment task or job 534 and/or other model related task or job may also be split into multiple smaller related tasks or jobs 532a-532d or 543a-543m for more efficient processing and use of the cloud computing infrastructure 522.

For example, the computing device 524 and/or cloud interface 528 (e.g. a Python API) may divide or split the ensemble generation task or job 526 into a plurality of model training tasks or jobs 526a, 526b, 526c, to 526n, where each model training task of the plurality of model training tasks or jobs 526a, 526b, 526c, to 526n is associated with a model of the plurality of models and a dataset of the plurality of datasets associated with compounds. Each of the model training tasks or jobs 526a, 526b, 526c, to 526n are submitted to the plurality of servers of the cloud computing infrastructure 522 for training the model corresponding to said each model training task or job.

Each of the tasks or jobs 526a, 526b, 526c, to 526n may be based on, by way of example only but is not limited to, a single input dataset of the plurality of datasets for training a single model of the plurality of models over a set of hyperparameters. Thus, the ensemble generation task of job 526 may be divided or split into multiple parallel model training tasks or jobs 526a, 526b, 526c, to 526n that each tackle the optimisation of a particular model in relation to a particular training dataset over a corresponding set of hyperparameters for the particular model. Each of the model training tasks or jobs 526a, 526b, 526c, to 526n may be different to avoid duplication of effort in finding the best trained models and corresponding datasets and hyperparameters. The cloud interface 528 may submit the individual jobs 526a, 526b, 526c, to 526n to the cloud computing infrastructure 522 (e.g. a train job or a deploy job etc.)

Each of the model training tasks or jobs 526a, 526b, 526c, to 526n and/or 532a-532b may calculate model performance statistics for the associated trained model, which may be sent to computing device 524. Computing device 524 may receive from each of the plurality of model training tasks or jobs 526a, 526b, 526c, to 526n and/or 532a-532b, the calculated model performance statistics for selecting and storing a set of optimal trained model(s) from the trained models based on the calculated model performance statistics of each trained model as described with reference to FIGS. 2c to 2g. Each of the model performance statistics or results from the individual model training tasks or jobs 526a, 526b, 526c, to 526n and/or 532a-532b may be used to determine or assess the best performing models from the individual jobs 526a, 526b, 526c, to 526n and/or 532a-532b. Each individual model training task or job provides one or more trained models, where each of those trained models that are determined to be the best performing trained models or meet certain performance criteria as described with reference to FIGS. 2f and 2g (also referred to herein as an “optimal” trained model). A trained model is optimal in the sense that the model performance statistics and the like of the trained model has met certain predefined performance criteria or thresholds as described with reference to FIGS. 2a-2g; the term “optimal trained model” will be used to refer to such trained models.

The optimal trained models that are selected and data associated with the model (e.g. input dataset used for training, chemical or compound descriptors, hyperparameters used for the model, model results and the like) may be stored in a trained model file or set of linked trained model files for future deployment. In particular, each trained model of the set of optimal trained models may be stored in a file system as a model file or model file object that includes data representative of at least one or more from the group of: the trained model, hyperparameters associated with the trained model, dataset used for training the trained model, chemical or compound descriptor associated with the trained model, and model performance statistics.

Additionally or alternatively, an ensemble model may be formed from multiple models of the set of optimal trained model(s) in an ensemble model file or file object that may include data representative of at least one from the group of: the multiple models making up the ensemble model, the file objects associated with the multiple models, datasets used for training the multiple models, hyperparameters associated with each of the multiple models, model performance statistics of the ensemble model and/or multiple models.

A user can thus have access, via computing device 524, to all of the optimal trained models via the file system, and may select the models to use by selecting the model files or file objects. The user may customise the models to meet their needs or requirements for deployment. Similarly, ensemble models may also be stored in a trained model file or file object that includes links or data representative of the corresponding model files of the models used in the ensemble model. In this manner, a user can have access, via computing device 524, to all of the models within the ensemble model, and may customise the models accordingly when deploying the ensemble model. A user may also create or generate further ensemble models by selecting two or more trained model files, the corresponding datasets/descriptors that will form the ensemble model, which may be saved in a trained model file corresponding to the ensemble model created.

In another example, a user may deploy one or more trained models for modelling a particular problem, process and the like by selecting from a set of trained model files one or more of the optimal models. The optimal models may be selected based on model type, chemical descriptor, and hyperparameters and other data and the like that may be described in each trained model file. The user may also specify the input dataset required for each of the selected models to operate on. The user's computing device 524 may then split or divide the selected models into multiple modelling tasks or jobs 532c-532d, in which each of the modelling tasks or jobs 532c-532d corresponds to one of the selected models. The input dataset for each of the modelling tasks or job 532c-532d can be generated in a similar manner as described with reference to FIGS. 2a and 2b. The input dataset for each modelling task or job may be generated based on a single input dataset that is replicated for each modelling task or job, but in which the chemical or compound descriptors of the single input dataset are replaced with the chemical or compound descriptor associated with the optimal model of that modelling task or job. Each generated input dataset may be incorporated into each modelling task or job for input to the trained optimal model.

Once the modelling tasks and jobs 532c-532d have been configured, the computing device 524 may submit, via the cloud interface 528 and communication network 530, the modelling tasks or jobs 532c-532d to the cloud computing infrastructure 522. The modelling tasks or jobs 532c-532d are dynamically allocated to one or more of the plurality of servers for processing. The results from each of the modelling tasks or jobs 532c-532d may be sent or received by the cloud interface 528 and presented to the computing device 524 for further review by the user etc. Each task may complete in its own time and is not dependent on any of the other tasks finishing or completing before results are provided to computing device 524. Once all tasks have finished, the results may be collated by the computing device 524. Alternatively or additionally, each of the modelling tasks or jobs 532c-532d may send their results and/or interim results to a results monitoring task or job (not shown), which may be configured for aggregating and/or combining the results from each of the modelling tasks or jobs 532c-532d. The results monitoring task or job may send the finalised results to the computing device 524 via the cloud interface 528 once all tasks have completed and results been combined and aggregated.

In another example, the user may deploy a predefined ensemble model that has been stored in the file system as an ensemble file object or file. The computing device 524 may generate an ensemble modelling task or job 534 by retrieving and configuring the models associated with the predefined ensemble model. The computing device or cloud interface 530 may split the ensemble modelling task or job 534 into a plurality of modelling tasks 534a-534m associated with the predefined ensemble model. Alternatively or additionally, the user may generate an ensemble model based on selecting a subset of the stored plurality of optimal trained models. In a similar manner, in which reference numerals are reused for simplicity, the computing device 524 may generate an ensemble modelling task or job 534 by retrieving and configuring the selected subset of models from the corresponding trained model files or file objects and the like. The computing device 524 or cloud interface 530 may split the ensemble modelling task or job 534 into a plurality of modelling tasks 534a-534m associated with the created ensemble model.

In any event, the computing device 524 or cloud interface 528 may further configure each of the modelling tasks or jobs 534a-534m of the ensemble modelling task 534 by generating an input dataset for each of the modelling tasks or jobs 534a-534m in a similar manner as described with reference to FIGS. 2a and 2b. For example, the input dataset for each modelling task or job may be generated based on a single input dataset that is replicated for each modelling task or job, but in which the chemical or compound descriptors of the single input dataset are replaced with the chemical or compound descriptor associated with the optimal model of that modelling task or job to form the input dataset for that optimal model. Each generated input dataset may be incorporated into each modelling task or job for input to the corresponding trained optimal model.

Once the modelling tasks and jobs 534a-534m have been configured, the computing device 524 may submit, via the cloud interface 528 and communication network 530, the modelling tasks or jobs 534a-534m of the ensemble model to the cloud computing infrastructure 522. The modelling tasks or jobs 534a-534m are dynamically allocated to one or more of the plurality of servers for processing. The results from each of the modelling tasks or jobs 534a-534m may be sent or received by the cloud interface 528 and presented to the computing device 524 for further aggregation, collation by an ensemble result task and/or review by the user etc. Each task may complete in its own time and is not dependent on any of the other tasks finishing or completing before results are provided to computing device 524. Once all tasks have finished, the results may be aggregated and/or collated by the computing device 524. Alternatively or additionally, each of the modelling tasks or jobs 534a-534m of the ensemble model may send their results and/or interim results to a results monitoring task or job (not shown), which may be configured for aggregating and/or combining the results from each of the modelling tasks or jobs 534a-534m. The results monitoring task or job may send the finalised results to the computing device 524 via the cloud interface 528 for review or interpretation for the user once all tasks have completed and results have been combined and/or aggregated.

Essentially, splitting the ensemble generation task/job 526 into multiple individual training model tasks or jobs 526a, 526b, 526c, to 526n, or individual model training tasks/jobs into multiple model training tasks or jobs 532a-532b, or the ensemble modelling task/job 534 into multiple individual modelling tasks or jobs 534a-534m, and/or individual modelling tasks/jobs into multiple modelling tasks or jobs 532c-532d can allow the user to customise a job then submit it to the cloud computing infrastructure 522 as opposed to the cloud-based system 500 of FIG. 5a, which may only processes entire ensemble generation task/jobs 506 and/or an ensemble modelling task (not shown). Although both systems 500 and 520 may have the same or similar functionality, the system 520 provides a more efficient use of computing resources by not requiring a dedicated set of computing resources to be on standby for processing large tasks/jobs 506. Furthermore, in the system 520, a user or automated monitoring process may also cull or terminate a particular individual job of the plurality of model training tasks or jobs 526a, 526b, 526c, to 526n and/or individual model training tasks/jobs into multiple model training tasks or jobs 532a-532b depending on the perceived performance of that particular individual job during training. Similarly, this may be applied to the plurality of modelling tasks 534a-534m and/or individual modelling tasks/jobs into multiple modelling tasks or jobs 532c-532d. This provides for further efficient processing by allowing the computing resources of the plurality of servers of cloud computing infrastructure 522 to be released as early as possible, which may then be used for other jobs and/or released altogether. Such efficient use of computing resources may also reduce the costs of operating and/or leasing the cloud computing infrastructure 522 and allow other users and/or computing devices to also submit ensemble models and the like for modelling their particular problems and/or processes and the like.

FIG. 5c illustrates a schematic diagram of an example model file storage system 540 for one or more models generated or used by example systems 500 and 520 of FIG. 5a and/or 5b. The file storage system 540 may include a data file storage unit 542 and a model file storage unit 546 for storing input datasets 542a-542d and/or model files 548 and/or 550 defining one or more trained models and the like, respectively. The model files may be managed and/or organised, by way of example only but is not limited to, in a loose database or a filesystem, which may be easily browsed by a user for retrieval of the trained model and the like for processing/modelling input datasets and the like. The data file storage unit 542 may be used to store a plurality of data files or input datasets 542a-542d. The data file storage unit 542 may use versioned data files for use in training one or more models and/or for input to one or more trained models. The input datasets 542a-542d may be used for training one or more models (e.g. labelled training datasets) such as, by way of example only but not limited to, ensemble generation task or job 506 as described with system 500 of FIG. 5a and/or ensemble generation task or job 526 comprising model training tasks or jobs 526a, 526b, 526c, to 526n, and/or model training tasks or job 532a-532b as described with system 520 of FIG. 5b. Alternatively of additionally, the input datasets 542a-542d may be used for input to one or more trained models (e.g. input datasets for processing by a trained model) as input datasets (e.g. input datasets for processing or modelling by trained models) such as, by way of example only but not limited to, input for modelling tasks 532a-532d and/or ensemble modelling task or job 534 comprising modelling tasks 534a-534m as described with system 520 of FIG. 5b. In this example, a model generation task or job 544 (e.g. ensemble generation task or job 506 or 526 of FIG. 5a or 5b, or model training task 532a-532b of FIG. 5b) is illustrated as receiving one or more input datasets 542a-542c for training one or more models associated with model generation task of job 544, for example, as described with reference to FIGS. 5a and/or 5b.

Once trained, the one or more trained models may be stored in a model file storage unit 546 in the form of model files 548 and 550. Each model file 548 or 550 may be a file object or file and is configured to include all the information about the trained model that enables a user to understand where it came from, how it was trained, the input datasets 542a-542d the model was trained on, model performance statistics and the like. Individual models may be stored in model files (e.g. model file 548) and/or ensemble models may be stored in ensemble model files (e.g. ensemble model file 550). For example, after an ensemble model has been generated (e.g. once ensemble generation task or job 506 or 526 of FIG. 5a or 5b, or model training task 532a-532b of FIG. 5b have completed), as described with reference to FIGS. 2a-5b, the multiple trained models and hyperparameters of the ensemble may be assessed, in which the best or optimal trained models may be selected, and the ensemble model stored and/or saved in a ensemble file object or ensemble model file 550 that includes data representative of all the selected models from each job or task, all associated optimised hyperparameters for each selected model, and/or model performance statistics and the like for forming or creating the ensemble model. Alternatively or additionally, each selected model may be stored in a separate model file object or file 548 and may be referred to by the ensemble model file and the like.

For example, model file 548 may include, by way of example only but is not limited to, data representative of the type of model 548a or ML technique used to train the model (e.g. random forest (RF), neural network (NN), LSTM, or other model), the model parameters and/or hyperparameters 548b for defining the model 548, one or more input datasets 548c (e.g. one or more of datasets 542a-542d), data featurisation method(s) 548d and/or model results/model performance statistics 548e providing further information on the trained model for assessment and possible selection by a user or model assembling/creation process. For example, model file 548 may include, by way of example only but is not limited to, data representative of the type of model 548a or ML technique used to train the model (e.g. random forest (RF), neural network (NN), LSTM, or other model), the model parameters and/or hyperparameters 548b for defining the model 548, one or more input datasets 548c (e.g. one or more of datasets 542a-542d), data featurisation methods (548d) and/or model results/model performance statistics 548e.

For example, ensemble model file 550 may be generated based on training a plurality of models or selecting a plurality of trained models. The ensemble model file 550 may include, by way of example only but is not limited to, data representative of the type of models and/or links to model files 550a that are combined together to form the ensemble model 550 (e.g. ML technique used to train the model such as, by way of example only but not limited to, random forest (RF), neural network (NN), LSTM, or other model), the ensemble model parameters and/or hyperparameters 550b for defining the ensemble model 550, which may define how the model files or models are combined to create the ensemble model (this may further include the hyperparameters of each individual model making up the ensemble model and the like), one or more input datasets 550c (e.g. one or more of datasets 542a-542d used for training the models used in the ensemble model), data featurisation method(s) 550d and/or ensemble model results/ensemble model performance statistics 550e providing further information on the trained model for assessment and possible selection by a user or model assembling/creation process.

In essence, data management for trained models and/or ensemble models in model files or file objects 548 or 550 allows any data or model data associated with the model to follow each trained model or ensemble model as it gets stored within the model file 548 or ensemble model file 550 itself. This avoids complex or centralised databases, where it is unclear what data item relates to which trained model and the like. As each model file 548 or 550 is stored in a file system 546, a user or other process may be able to open the model file and view one or more trained models, datasets, hyperparameters, etc., that are contained therein. The model file 548 or 550 is configured to store the model information and “experiments” on how it is trained, as well as the trained parameters defining the model etc. Ensemble model file or file structures 550 may also contain multiple files of models or links to the multiple model files defining the ensemble model, and may each include an additional file on how they are all combined. Thus a user or other process may be able to assess each model by reading the corresponding model file and determine how it was trained and also the model performance statistics, weaknesses and/or strengths of the model for modelling certain datasets 542a-542d and the like. Thus, all model information associated with a model may be stored in a model file 548 or 550 from training through to deployment and the like. That is the model information is added to the model file 548 and/or 550 as it proceeds along the model training pipeline and/or deployment processing pipelines.

FIG. 5d is a schematic diagram illustrating an example model report file or file object structure 560 for either an ensemble model and/or individual trained model according to the invention. Every trained model that is stored in the model file storage unit 546 may include a model report file or file object structure 560 that a user or process may read and/or browse to assess the corresponding trained model(s) therein. The model report file may be based on a mark-up language such as, by way of example, hypertext mark-up language (HTML), in which a web browser may display model data report associated with the trained model file (e.g. model file 548 or 550) stored in model file storage unit 546.

The model report file 560 includes data representative of the type of model and/or links to models 560a. In this example, the model report file 560 describes the type of model is by the character string “model name”: “rf”, which indicates the ML technique used to train the model as a random forest ML technique. The model report file 560 also includes, by way of example only but not limited to, the model parameters and/or hyperparameters 560b that were used to train the model. The model report file 560 may also include data representative of the training dataset and/or input dataset (e.g. labelled training dataset) which may include, by way of example only but not limited to, filenames, links and or file paths directed to the input datasets (e.g. in this case a file path may be used to indicate what labelled initial training input dataset was used, which is indicated by the character string ““data_path”: “/Users/userxy/data/BBBP/BBBP_updated.csv”), the types of compound descriptors the training dataset is based on may also be described (e.g. the compound descriptor SMILES is indicated by the character string ““feature keys”: [“SMILES”]”), output filenames, links and or file paths directed to the output or result datasets (e.g. in this case a file path may be used to indicate what output/result dataset may be or was used, which is indicated by the character string ““output_dir”: “/Users/userxy/data/BBBP/”), and any other input and/or output datasets and information thereto. The model report file may also include data representative of featurization methods 560d and the like (e.g. this may be represented by the character string” “featurizers”:[“morgan_2048_counts”]”). In addition to the model type 560a, model parameters and/or hyper parameters 560b, datasets 560c, and/or featurization methods 560d, the model training results and/or performance statistics 560e may be described including data representative of the overall performance of the trained model defined in model report file 560. The model performance statistics 560e may include performance data and/or statistics associated with prediction and/or recall accuracy and the like as described with reference to FIGS. 1a to 5b, which may include, by way of example only but is not limited to, area under the curve (AUC), area under the precision recall curve (AUprC), F1 score, precision, recall, accuracy, sensitivity, and/or specificity and the like, r2 (r squared), root mean squared error (RMSE), mean squared error (MSE), median absolute error, mean absolute error, Matthews correlation coefficient (MCC), model Accuracy, model precision, model recall and the like, combinations thereof, modifications thereof and/or any other model performance statistic or results thereto for use in assessing the performance of training a model and the resulting trained model on test datasets and the like. In this example, the model report file 560 indicates the overall performance 560e resulting from training the model and also testing the trained model based on a table with columns related to results during testing the trained model (e.g., column “Test”), and results from training the model (e.g. column Train), with rows related to various model performance statistics including, by way of example only but not limited to, model performance statistics and/or results based on MCC, Accuracy, Precision, Recall and F1. Furthermore, the overall performance 560e may also indicate, by way of example only but not limited to, data representative of the best predictions and worst predictions. In this case, data representative of the best predicted molecules may be indicated and also data representative of the worst predicted molecules may be indicated. Thus, the model report file 560e may show performance of model, molecules best predicted, molecules worst predicted, chemical structures and information. The model report file 560 may be read and displayed as by a graphical user interface (GUI) as a visualisation to assist users to understand a trained model in a model file selected from the model file storage unit 546. For example, the GUI visualisation may be configured to allow users to hover over the table and display performance graphs, show pictures of worst and best performing molecules, display structural representation of the trained model based on hyperparameters and the like, etc.

The embodiments described above can be fully automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device, apparatus or any of the functionality that is described herein may be performed on a distributed computing system, such as, by way of example only but not limited to one or more server(s), one or more cloud computing system(s). Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements. As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method of generating an ensemble model, the method comprising:
training a plurality of models based on a plurality of datasets associated with compounds;
calculating model performance statistics for each of the plurality of trained models;
selecting and storing a set of optimal trained model(s) from the trained models based on the calculated model performance statistics; and
forming one or more ensemble models, each ensemble model comprising multiple models from the set of optimal trained model(s).

2. A computer-implemented method according to claim 1, wherein calculating model performance statistics further comprises cross-validating each of the plurality of models.

3. A computer-implemented method according to claim 1, wherein calculating the model performance statistics for each trained model comprises calculating at least one or more model performance statistics for each trained model based on one or more from the group of:
positive predictive value or precision of the trained model;
sensitivity, specificity, true predictive rate, or recall of the trained model;
a receiver operating characteristic, ROC, graph associated with the trained model;
an area under a ROC curve associated with the trained model;
an area under a precision ROC curve associated with the trained model;
an area under a precision and recall ROC curve associated with the trained model;
F1 score;
r-squared;
root mean squared error;
mean squared error;
median absolute error;
mean absolute error;
any other function associated with precision and/or recall of the trained model; and
any other model performance statistic(s) for evaluating each of the trained models based on model type or machine learning (ML) technique associated with each model.

4. A computer-implemented method according to claim 1, wherein the method further comprises: generating a plurality of datasets from a set of labelled datasets associated with compounds.

5. A computer-implemented method according to claim 4, wherein generating the plurality of datasets further comprises generating groups of datasets from the set of labelled datasets based on a plurality of compound descriptors, wherein each group of datasets corresponds to a different compound descriptor.

6. A computer implemented method according to claim 5, wherein a compound descriptor comprises a compound descriptor based on at least one or more of:
International Chemical Identifier, InChI;
InChIKey;
MoIFile format;
two dimensional Physical Chemical descriptors;
three dimensional Physical Chemical descriptors;
XYZ file format;
Extended Connectivity Fingerprint, ECFP;
Structure Data Format;
structural formula or representation of the compound;
Simplified Molecular Input Line Entry Specification, SMILES, strings or format;
SMILES arbitrary target specification or format;
Chemical Mark-up Language format; and
any other chemical descriptor or chemical descriptor format for describing, representing and/or encoding molecular information and/or structure(s) of compounds.

7. A computer-implemented method according to claim 4, wherein:
generating the plurality of datasets further comprising generating, for each dataset of the plurality of datasets, a set of dataset folds by partitioning said each dataset into multiple portions; and
for the plurality of models and the plurality of datasets, performing the steps of:
training each model based the set of dataset folds corresponding to each dataset;
calculating model performance statistics for each trained model based on each fold of the set of dataset folds corresponding to each dataset; and
storing data representative of the trained model in a set of optimal models based on the calculated model performance statistics.

8. A computer implemented method according to claim 7, wherein storing data representative of the trained model further comprises storing data representative of the trained model in the set of optimal models by comparing the calculated model statistics with one or more performance thresholds associated with the model statistics.

9. A computer implemented method according to claim 7, wherein storing data representative of the trained model further comprises storing data representative of the trained model in the set of optimal models by comparing the calculated model statistics with the calculated model statistics of previously stored models.

10. A computer implemented method according to claim 9, further comprising deleting previously stored models from the set of optimal models based on the calculated model statistics of a model of the same type.

11. A computer-implemented method according to claim 7, wherein storing data representative of the trained model further comprises storing data representative of the trained model, the calculated model statistics of the trained model, and/or the dataset associated with training the trained model.

12. A computer-implemented method according to claim 7, further comprising repeating the steps of training, calculation and storing for each of a set of hyperparameters selected from a plurality of hyperparameters associated with said each model.

13. A computer-implemented method according to claim 7, wherein the plurality of models further comprises models configured based on a set hyperparameters selected from a plurality of hyperparameters associated with each type of model of the plurality of models.

14. A computer-implemented method according to claim 1, wherein forming one or more ensemble of models further comprises selecting a subset of optimal models from the set of optimal model(s), wherein each model in the subset of optimal models has improved model statistics compared with the remaining models in the set of optimal models.

15. A computer-implemented method according to claim 14, wherein selecting a subset of optimal models from the set of optimal model(s) further comprises ranking the optimal models based on the model statistics and selecting a subset of the topmost ranked optimal models for inclusion into the ensemble model.

16. A computer-implemented method according to claim 14, wherein selecting a subset of optimal models from the set of optimal model(s), further comprises:
retrieving models and associated model statistics from the set of optimal models that correspond to the same model type;
ranking the retrieved models based on the model statistics; and
selecting one or more model(s) from the retrieved models having the highest model statistics for inclusion into the ensemble model.

17. A computer-implemented method according to claim 14, wherein selecting a subset of optimal models from the set of optimal model(s), further comprises, for each of the plurality of datasets:
retrieving the models and associated model statistics from the set of optimal models that are associated with the same dataset;
ranking the retrieved models based on the model statistics; and
selecting one or more topmost model(s) from the ranked retrieved models for inclusion into the ensemble model.

18. A computer-implemented method according to claim 1, further comprising benchmarking the one or more ensemble models based on the plurality of datasets.

19. A computer-implemented method according to claim 18, wherein benchmarking the one or more ensemble models further comprises calculating ensemble model statistics based on cross-validating each of the one or more ensemble models.

20. A computer-implemented method for using an ensemble model, wherein the ensemble model is based on an ensemble model generated according to claim 1, the method comprising:
inputting, to the ensemble model, data representative of one or more labelled dataset(s) used to generate and/or train the model(s) of the ensemble model; and
receiving, from the ensemble model, output data associated with labels of the one or more labelled dataset(s).

21. A computer-implemented method for modelling a process or problem associated with compound(s), the method comprising:
inputting, to an ensemble model for modelling the process or problem, representations of one or more compound(s);
receiving, from the ensemble model, results associated with modelling the process or problem based on the one or more compound(s); and
wherein the ensemble model comprises multiple model(s) automatically selected based on model performance statistics calculated for each of the model(s).

22. An apparatus comprising a processor, a memory unit and a communication interface, wherein the processor is connected to the memory unit and the communication interface, wherein the processor and memory are configured to implement the computer-implemented method according to claim 1.

23.-28. (canceled)

29. A tangible computer-readable medium comprising computer executable instructions, which when executed by one or more processor(s), causes at least one of the one or more processor(s) to perform at least one of the steps of the method of:
training a plurality of models based on the plurality of datasets associated with compounds;
calculating model performance statistics for each of the plurality of trained models;
selecting and storing a set of optimal trained model(s) from the trained models based on the calculated model performance statistics; and
forming one or more ensemble models, each ensemble model comprising multiple models from the set of optimal trained model(s).

30. The computer-readable medium according to claim 29, wherein when executed on the processor, the computer executable instructions cause the processor to implement the computer-implemented method of claim 2.

31. An apparatus comprising a processor and a memory unit, the processor is connected to the memory unit, wherein:
the processor is configured to train a plurality of models based on a plurality of datasets associated with compounds;
the processor is configured to calculate model performance statistics for each of the plurality of trained models;
the processor and memory are configured to selecting and storing a set of optimal trained model(s) from the trained models based on the calculated model performance statistics; and
the processor and memory are configured to form one or more ensemble models, each ensemble model comprising multiple models from the set of optimal trained model(s).

32. An apparatus comprising a processor, a memory unit and a communication interface, the processor is connected to the memory unit and the communication interface, wherein:
the processor and communication interface are configured to retrieve an ensemble model generated according to claim 1,
the processor and memory are configured to input, to the ensemble model, data representative of one or more labelled dataset(s) used to generate and/or train the model(s) of the ensemble model; and
the processor and memory are configured to receive, from the ensemble model, output data associated with labels of the one or more labelled dataset(s).

33. An apparatus comprising a processor, a memory unit and a communication interface, the processor is connected to the memory unit and the communication interface, wherein:
the processor is configured to input, to an ensemble model for modelling a process or problem associated with compounds, representations of one or more compound(s);
the processor and memory are configured to receive, from the ensemble model, results associated with modelling the process or problem based on the one or more compound(s); and
wherein the ensemble model comprises multiple model(s) automatically selected based on model performance statistics calculated for each of the model(s).

34. A system for generating an ensemble model, the system comprising:
a dataset generation module configured for generating a plurality of datasets associated with compounds based on multiple labelled datasets;
a model generation module configured to train a plurality of models based on the plurality of datasets associated with compounds, wherein model performance statistics are calculated for each of the plurality of trained models;
a model selection module configured to select and store a set of optimal trained model(s) from the plurality of trained models based on the calculated model performance statistics; and
a ensemble creation module configured to retrieve multiple models from the set of optimal trained models and form one or more ensemble models, each ensemble model comprising multiple models from the set of optimal trained model(s).

35. The system of claim 34, further comprising:
an ensemble benchmark module configured to retrieve a formed ensemble model and benchmark the retrieved ensemble model based on the corresponding plurality of datasets used to generate each of the models forming the ensemble model; and
an ensemble database module configured to store the benchmarked ensemble models and benchmark results.

36. (canceled)

37. A computer-implemented method according to claim 1, further comprising stacking each ensemble model using a combiner ML technique to generate, based on labelled training datasets of the models of the ensemble model, a combiner ML model for combining the predictions or outputs from each of the models to form a data representative of a final prediction or final data output of the ensemble model.

38. A computer-implemented method according to claim 1, wherein training the plurality of models further comprises splitting the ensemble generation into a plurality of model training tasks or jobs, wherein each model training task is associated with a model of the plurality of models and a dataset of the plurality of datasets associated with compounds; and submitting each model training task or job to a plurality of servers for training the model associated with said each model training task or job.

39. A computer-implemented method according to claim 38, wherein each of the model training tasks or jobs calculate model performance statistics for the associated trained model, and, receiving from each of the plurality of model training tasks or jobs, the calculated model performance statistics for selecting and storing a set of optimal trained model(s) from the trained models based on the calculated model performance statistics of each trained model.

40. A computer-implemented method according to claim 39, further comprising storing each trained model of the set of optimal trained models in a model file object including data representative of at least one or more from the group of: the trained model, hyperparameters associated with the trained model, chemical or compound descriptor associated with the trained model, dataset used for training the trained model, and model performance statistics.

41. A computer-implemented method according to claim 40, further comprising storing each ensemble model formed from multiple models of the set of optimal trained model(s) in a ensemble model file object including data representative of at least one from the group of: the multiple models, the file objects associated with the multiple models, datasets used for training the multiple models, hyperparameters associated with each of the multiple models, model performance statistics of the ensemble model and/or multiple models.

42. A computer-implemented method according to claim 38, wherein each ensemble training task or job further includes a set of hyperparameters associated with the model.
